(19) United States
(12) Patent Application Publication
(10) Pub. No.: US 2013/0183345 A1
(43) Pub. Date: Jul. 18, 2013

(54) Treatment of Bladder Cancer Following Detection of Expression Levels of Certain Progression Markers

(71) Applicants: Lars Dyrskjøt Andersen, Odder (DK); Torben Falck Orntoft, Silkeborg (DK); Joseph A. Sorge, Wilson, WY (US); Alexey Novoradovsky, San Diego, CA (US)

(72) Inventors: Lars Dyrskjøt Andersen, Odder (DK); Torben Falck Orntoft, Silkeborg (DK); Joseph A. Sorge, Wilson, WY (US); Alexey Novoradovsky, San Diego, CA (US)

(73) Assignees: CATALYST ASSETS, LLC, Jackson, WY (US); AAB PATENT HOLDING

(21) Appl. No.: 13/791,370

(22) Filed: Mar. 8, 2013

Related U.S. Application Data
(63) Continuation-in-part of application No. 13/323,554, filed on Dec. 12, 2011.
Application No. 13/323,554 is a continuation-in-part of application No. 12/180,321, filed on Jul. 25, 2008.
Application No. 12/180,321 is a continuation of application No. 10/533,547, filed on Nov. 16, 2005, now abandoned.
Application No. 10/533,547 is a continuation-in-part of application No. 13/316,733, filed on Dec. 12, 2011.
(60) Provisional application No. 61/559,652, filed on Nov. 14, 2011.

(51) Int. Cl. A61K 35/74

(52) U.S. Cl. CPC A61K 35/74
USPC 424/246.1

(57) ABSTRACT

Disclosed is determining expression levels of protective or harmful markers for bladder cancer prognosis and treatment; particularly, determining the expression levels of protective markers (COL4A3BP, MBNL2, FABP4, NEK1 and SKAP2) and harmful markers (COL4A1, UBE2C, BIRC5, COL18A1, KPNA2, MSN, ACTA2, and CDC25B) and making treatment decisions in consideration of increased or decreased risk of progression based on the marker expression levels.

### RELATED APPLICATIONS

This Application is a continuation-in-part of U.S. application Ser. No. 13/316,733, filed Dec. 12, 2011 (pending), which is a non-provisional of Provisional No. 61/559,652, filed Nov. 14, 2011 (expired), and this application is also a continuation-in-part of U.S. application Ser. No. 13/323,554, filed Dec. 12, 2011 (pending), which is a continuation-in-part of U.S. application Ser. No. 12/180,321, filed Jun. 25, 2008 (pending), which is a continuation of U.S. application Ser. No. 10/533,547, filed Nov. 16, 2005 (abandoned), which is a National Stage Entry of PCT/DK03/00750, filed Nov. 3, 2003 (expired), which claims priority to Danish Application No. PA 2002 01685, field 11-01-2002 (expired).

### SEQUENCE LISTING

This application contains a Sequence Listing which has been submitted in ASCII format via EFS-Web and is hereby incorporated by reference in its entirety. Said ASCII copy, created on Mar. 1, 2013, is named SRG-COL4-CIP_SL.txt and is 92,141 bytes in size.

### FIELD OF THE INVENTION

The invention relates to determining expression levels of genes or markers where the expression levels have been determined to correlate with progression or non-progression of bladder cancer.

### BACKGROUND

In industrialized countries, urinary bladder cancer is the fourth most common malignancy in males, and the fifth most common neoplasm overall. A total of 70,530 new cases and 14,680 deaths were estimated in the US alone in 2010 (Jemal A, et al., Cancer statistics, 2010. CA Cancer J Clin; 60: 277-300). The disease basically takes two different courses; one where patients have multiple recurrences of superficial tumors (Ta and T1), and one where tumors progress to a muscle invasive form (T2+) which can lead to metastasis. About 5-10% of patients with Ta tumors and 20-30% of the patients with T1 tumors will eventually develop a higher stage tumor (Wolf H, et al., Bladder tumors, Prog Clin Biol Res 1986; 221:223-55). More than 60% of patients with non-muscle invasive bladder tumors experience bladder tumor recurrences and around 20% of the patients develop disease progression to a muscle-invasive bladder cancer (Millan-Rodriguez F, et al., Primary superficial bladder cancer risk groups according to progression, mortality and recurrence. J Urol 2000; 164: 680-4 and Sylvester R J, et al. Predicting recurrence and progression in individual patients with stage Ta T1 bladder cancer using EORTC risk tables: a combined analysis of 2596 patients from seven EORTC trials: Eur Urol 2006; 49: 466-5; discussion 75-7). Patients with superficial bladder tumors represent 75% of all bladder cancer patients. No approved clinically useful markers separating such patients by likelihood of progression exist (Ehadie B, et al. Predicting tumor outcomes in urothelial bladder carcinoma: turning pathways into clinical biomarkers of prognosis. Expert Rev Anticancer Ther 2008; 8: 1103-10).

It is believed that patients presenting with isolated or concomitant carcinoma in situ (CIS) lesions have a higher risk of disease progression to a muscle invasive stage. The CIS lesions may have a widespread manifestation in the bladder (field disease) and are believed to be the most common precursors of invasive carcinomas. See Spruck, C. H., et al. Two molecular pathways to transitional cell carcinoma of the bladder, Cancer Res. 54: 784-788, 1994; Rosin, M. P. et al. Partial allelotype of carcinoma in situ of the human bladder. Cancer Res, 55: 5213-5216 1995. Other clinical risk factors associated with a high risk of disease progression to a muscle invasive cancer include deep invasion of the lamina propria, high grade tumor, large tumor size, tumor multiplicity, and recurrence of high risk non-muscle invasive tumors (Hermann G G, et al., The influence of the level of lamina propria invasion and the prevalence of p53 nuclear accumulation on survival in stage T1 transitional cell bladder cancer. J Urol 1998; 159: 91-4). Generally, it is known that stage T1 tumors have a higher risk of further progression than stage Ta tumors. The ability to predict which tumors are likely to recur or progress would have great impact on the clinical management of patients with superficial disease, as it would be possible to treat high-risk patients more aggressively (e.g. with radical cystectomy or adjuvant therapy). Clinical risk factors cannot predict individual disease course and the recurrent nature of bladder cancer makes it one of the most expensive cancers to treat (Avritscher E B, et al., Clinical model of lifetime cost of treating bladder cancer and associated complication. Urology 2006; 68: 549-53), thus there is a great need for molecular markers capable of predicting the risk of bladder tumor recurrence or later disease progression.

Although many prognostic markers have been investigated, the most important prognostic factors are still disease stage, dysplasia grade, and especially the presence of areas with CIS. See Anderstrom, et al., The significance of lamina propria invasion on the prognosis of patients with bladder tumors. J Urol. 124:23-26, 1980; Cummings, K. B. Carcinoma of the bladder: predictors. Cancer, 45:1849-1855, 1980. Cheng, L. et al., Survival of patients with carcinoma in situ of the urinary bladder. Cancer, 85:2469-2474, 1999. The standard for detection of CIS is histopathologic analysis of a set of selected site biopsies removed during routine cystoscopy examinations, often in combination with 5-ALA fluorescence imaging of the tumors and pre-cancerous lesions (CIS lesions and moderate dysplasia lesions) Kriegmair, M. et al., Early clinical experience with 5-aminolevulinic acid for the photodynamic therapy of superficial bladder cancer. Br J Urol. 77: 667-671, 1996.

Monitoring gene expression levels may be used to find indicator genes or indicator gene products also referred to herein as markers. One type are harmful markers, whose elevated expression correlates with bladder cancer progression or death from bladder cancer, and another type are protective markers, in the sense that their elevated expression levels correlate with a lower frequency of progression and a lower frequency of death from bladder cancer. Further, once such markers are found, one may combine the gene expression levels of the protective and harmful markers into sets or signatures, which, in combination, may indicate the likelihood of progression or bladder cancer death more reliably than when monitoring them separately.

Gene expression levels can be monitored by assaying a subject's mRNA using a method or process that detects a signal coming from the mRNA molecules. Examples of methods or processes used to monitor gene expression levels include nucleic acid hybridization, quantitative polymerase chain reaction (or other nucleic acid replication reactions), nucleic acid sequencing, protein product detection, and visible light or ultra-violet light spectrophotometry or diffraction. Such methods can utilize fluorescent dyes, radioactive tracers, enzymatic reporters, chemical reaction products, or other means of reporting the amounts or concentrations of nucleic acid molecules. Gene expression levels can be monitored by first reverse transcribing the mRNA from a subject's sample to produce cDNA, then amplifying the cDNA using the polymerase chain reaction (PCR). One preferred method of detecting gene expression levels is with reverse-transcriptase quantitative PCR (QRT-PCR).

### SUMMARY

The inventions described and claimed herein have many attributes and embodiments including, but not limited to, those set forth or described or referenced in this Summary. It is not intended to be all-inclusive and the inventions described and claimed herein are not limited to or by the features or embodiments identified in this Summary, which is included for purposes of illustration only and not restriction.

The invention relates to treatment of bladder cancer, where the treatment is tailored in accordance with the risk of progression of the bladder cancer. The risk of progression is determined by determining expression levels of certain markers for which increased expression indicates a favorable prognosis (referred to as favorable or protective markers), i.e., increased expression correlates with both lack of progression of the subject's bladder cancer beyond stage Ta or T1 and lack of death from bladder cancer, and decreased expression of these protective markers correlates with progression beyond stage Ta or T1 or death from bladder cancer. The risk of progression is also determined by determining expression levels of other markers for which increased expression indicates an unfavorable prognosis (referred to as unfavorable or harmful markers), i.e., increased expression correlates with progression of the subject's bladder cancer beyond stage Ta or T1 or death from it, and decreased expression correlates with lack of progression beyond stage Ta or T1 or death from bladder cancer. More particularly, increased expression of mRNAs or other gene products from COL4A3BP, optionally in combination with increased expression of MBNL2, FABP4, NEK1, and/or SKAP2 (protective markers), correlates with lack of progression of a subject's bladder cancer, and decreased expression levels of these mRNAs correlates with progression of bladder cancer. Increased expression of mRNAs or other gene products from COL4A1, optionally in combination with increased expression of UBE2C, BIRC5, COL18A1, KPNA2, MSN, ACTA2, and/or CDC25B mRNAs (harmful markers) correlates with progression of the subject's bladder cancer, and decreased levels correlate with lack of such progression. COL4A3BP, which is favorable or protective, was not previously known to be associated with bladder cancer progression.

Such increased or decreased expression levels of these markers are preferably determined relative to a cut-off value, which can be determined empirically, and which exact value will depend on the type of assay and instrumentation used to quantify concentrations or copy number. A cut-off value in one embodiment is the mean amount of marker measured in a collection of samples obtained from bladder cancer patients.

Detection of expression levels of some or all of these markers in early-stage bladder cancer patients can be used to predict patient outcomes and/or tailor treatments. Expression levels can be determined by measuring a gene product of a particular gene in a sample. Gene products include pre-mRNA, mRNA, cDNA transcribed from the mRNA, and protein translated from mRNA. A preferred technique for measuring the gene products present in a subject's samples includes QRT-PCR (quantitative reverse transcriptase polymerase chain reaction). Expression arrays, nucleic acid sequencing, fluorescent nucleic acid dyes and/or chelators can also be used to determine cDNA levels, as well as techniques for assaying for particular protein products, including ELISA, Western Blotting, and enzyme assays. These techniques for measuring expression levels are listed for illustrative purposes and are not meant to be limiting. Other methods will be apparent to those of skill in the art and any methods for measuring expression levels are within the scope of the inventions described herein.

In a preferred embodiment, the relative amount of one or more markers is determined relative to one or more other markers in the assay, and, more preferably, to one or more other markers in a progression signature. In a particularly preferred embodiment, markers that individually proved to be predictive of risk of bladder cancer progression are grouped into signatures. The expression levels of all favorable markers in the signature are averaged together and the expression levels of all unfavorable markers in the signature are averaged together, and then the difference between the two averaged expression levels, favorable and unfavorable, is determined (i.e., the harmful average is subtracted from the protective average). The difference between the averages is a measure of the relative amount of these favorable versus unfavorable markers in a sample, and can be used to more accurately predict the likelihood of progression or death from bladder cancer, than simply looking at expression levels of individual favorable or unfavorable markers in isolation.

In one embodiment, the expression levels of various markers are measured using quantitative PCR (QPCR), and determining the Ct values for these markers. The Ct value for a particular marker in one patient sample is compared to the Ct values for the same marker in a population of bladder cancer patient samples. If the Ct value of the individual patient sample falls above or below the mean or median of all Ct values for that marker in the population of bladder cancer samples then that patient is said to have either higher or lower expression of the marker. When this information is compared to the clinical data for the index patient, a determination can be made about the correlation of the expression level of the marker and clinically determined progression or non-progression of bladder cancer. This same method can be used to evaluate all markers for their correlation to progression of disease. Once two or more markers significantly correlated with progression or non-progression of bladder cancer have been identified these markers can be grouped into signatures comprising protective and harmful markers. Risk scores for bladder cancer progression can be calculated using Ct values according to the formula: average Ct (protective markers)−average Ct (harmful markers).

In monitoring expression levels with PCR-based assays, reducing noise relative to signal enhances assay reliability. There are many ways to reduce noise, which can be used in combination with the method set forth herein, and such combined methods are within the scope of the invention. Where one determines progression risk for a patient by forming a signature including favorable markers and unfavorable markers, averaging the expression levels of the favorable and unfavorable markers separately and subtracting the averages, as described above, such a method may in itself reduce noise. For example, in a signature comprised of favorable and unfavorable markers where signals (measured for example by Ct value) from the unfavorable markers are subtracted from the signals for the favorable markers (as described above), this subtraction step can act to reduce noise relative to the signal. This noise reduction step is also an embodiment of the invention, and can be applied to reduce noise relative to signal in virtually any type of DNA, mRNA or protein assay wherein a signal is measured or monitored. It eliminates the need to normalize Ct values or other measures of target signal.

In one embodiment receiver operator characteristic (ROC) curves are used to determine the cut-off values. The optimal cut-off value providing the risk score with the highest sensitivity and specificity or, e.g. a 90% sensitivity cut-off value, could both be identified using the area under the curve (AUC) from the ROC curves. This would be delineated by analyzing a number of samples with known progression and a number of samples with no disease progression. The optimal cut-off value as determined by AUC in an ROC plot will be influenced by various clinical and monetary concerns. There is always a balance between the negative impact of missing true positive samples and misdiagnosing false positive samples. Depending on whether it is more beneficial to have a certain number of patients who, for example, are unlikely to progress to more advanced bladder cancer be identified as progressors; or whether it is more beneficial to have a certain number of patients who, for example, are likely to progress to more advanced bladder cancer be identified as non-progressors, will influence where the optimal cut-off point is set. The optimal cut-off point will be determined by the consequences of making a false diagnosis.

In another embodiment signatures comprising two or more markers significantly correlated with clinically determined progression or non-progression of bladder cancer can be used to determine risk of bladder cancer progression along a continuum. Some patients will be classified as at high risk of progression, others will be identified as at intermediate risk and still others as at low risk of progression. Each of these classifications will have clinical consequences. For example high risk patients may be monitored for bladder cancer recurrence, metastasis or other form of progression more frequently; they may also be good candidates for cystectomy or other more aggressive treatment options. Low risk patients, may for example be monitored at slightly greater intervals, for example every four months rather than every two months. Intermediate risk patients might follow a more standard treatment and monitoring protocol because the signature would not place them into either high or low risk categories distinctly. Measuring the risk of progression from the signature can be based on the Ct values of the markers or ROC curves as described above or various other statistical analyses. Non-limiting examples of such analysis methods are Pearson correlation. Wilcoxon signed rank test, and Cox regression analysis. Any method for determining expression levels for markers in a signature may be employed in the calculations for assessing risk of progression.

In certain embodiments it may be useful to assign different significance or weight to particular harmful and protective markers in a signature used to make a determination about an individual's prognosis in a disease. For example, a signature comprising markers significantly correlated with risk of bladder cancer progression, may contain one or more markers whose expression levels are even more significantly correlated with risk of progression (Note: this can either be a decreased risk of progression as with protective markers or an increased risk of progression as with harmful markers) than the expression levels of other markers in the signature. Any marker(s) showing increased correlation with risk of bladder cancer progression compared to other markers in the signature could be weighted more heavily than those other markers in a manner that reflects their increased statistical correlation with the clinical outcome. One example of how this might be achieved is to look at a group of patients whose bladder cancer progressed and a second group of patients whose bladder cancer did not progress. Then for each group of patients weight the preferred protective markers, for example COL4A3BP and/or including MBNL2, FABP4, NEK1, and SKAP2; and weight the preferred harmful markers, for example COL4A1 and/or including any of UBE2C, BIRC5, COL18A1, KPNA2, MSN, ACTA2 and CDC25B. In each instance the objective of the weighting would be to achieve the best correlation with risk of bladder cancer progression in each patient group; high risk and low risk. The weights may be adjusted in many ways depending on the particular clinical needs at the time of assessment. For example, one may adjust the weighting to reduce the number of patients who are likely to progress being falsely categorized as at low risk of progression. Such patients might then consider more aggressive treatment regimens. Alternatively, the weighting can be adjusted to reduce the number of patients who are unlikely to progress being falsely categorized as at high risk of progression. Thus a reduced number of patients may receive aggressive treatment. It will be apparent to one of skill in the art that other clinical concerns could affect how particular markers are weighted and these methods are all included in this embodiment.

It is contemplated that one might use a Cox regression analysis to determine the independent contribution of the expression level of each marker in a signature to overall likelihood of bladder cancer progression. Each marker in a signature may contribute to the overall risk of progression differently or be weighted differently. One could use the Cox covariate regression analysis to determine the coefficient (i.e. weight) for each marker in the signature and this coefficient may be multiplied by the measure of the expression level for a particular marker such as, but not limited to, a Ct value to determine a score for the signature where individual markers are evaluated based upon the significance of the correlation of the expression levels for each individual marker to the risk of progression. In a signature composed of six markers, where some are protective and some are harmful, the calculation for score might look like:

Score=((a*Ct(PM1)+b*Ct(PM2)+c*Ct(PM3))/3)−((d*Ct(HM1)+e*Ct(HM2)+f*Ct(HM3))/3)

Or in a preferred alternative, one could calculate score by dividing the sum of the weighted Ct's (or other measure of expression levels) for the protective markers by the sum of the weights for each protective marker in the signature and then dividing the sum of the weighted Ct's (or other measure of expression levels) for the harmful markers by the sum of the weights for each harmful marker in the signature. Finally, you would subtract the score calculated for the harmful markers from the score calculated for the protective markers as shown below. Such a calculation would then allow one to subtract out much of the possible sources of noise in determining the expression levels for the protective and harmful markers of the signature.

Score=((a*Ct(PM1)+b*Ct(PM2)+c*Ct(PM3))/Σ(a,b,c))−((d*Ct(HM1)+e*Ct(HM2)+f*Ct(HM3))/Σ(d,e,f))

Where a-f are the coefficients (i.e. weights) determined by regression analysis;

PM1, PM2 and PM3 are protective markers; and

HM1, HM2 and HM3 are harmful markers.

Other statistical methods or analysis methods could be used to determine coefficients or weights for each marker. Other methods than determining Ct values could be used to determine the expression levels for each marker. The above calculations for score are just two possible methods for factoring in the possible differences in significance for each marker in a signature. Other methods will occur to those of skill in the art and are incorporated herein. It will be obvious that each marker in the progression signature may be equally significant in determining likelihood of progression and in which case all coefficients a-f will be the same.

### BRIEF DESCRIPTION OF THE FIGURES

FIG. 1 Kaplan Meier survival plot for a preferred gene signature that was found to be highly predictive of the likelihood of bladder cancer progression or non-progression comprising COL4A3BP, MBNL2, FABP4, COL4A1, and UBE2C. The upper line shows the patients with a low score for this signature, the lower line shows the patients with a high score for this signature and the middle line shows all patients together.

FIG. 2 Kaplan Meier survival plot for the harmful marker COLA-A1. The upper line shows the patients with low expression levels for COL4-A1 and the lower line shows the patients with high expression levels for COL4A1.

FIG. 3 Kaplan Meier survival plot for the protective marker COL4A3BP. The lower line shows the patients with low expression levels for COL4A3BP and the upper line shows the patients with high expression levels for COL4A3BP.

FIG. 4 Receiver Operating Characteristic (ROC) curves. FIG. 4A shows the ROC curve for the harmful marker COL4A1 after 24 months. FIG. 4B is after 36 months and FIG. 4C is after 60 months.

FIG. 5 Receiver Operating Characteristic (ROC) curves. FIG. 5A shows the ROC curve for the protective marker COL4A3BP after 24 months. FIG. 5B is after 36 months and FIG. 5C is after 60 months.

FIG. 6 Receiver Operating Characteristic (ROC) curves. FIG. 6A shows the ROC curve for a preferred gene signature that was found to be highly predictive of the likelihood of bladder cancer progression or non-progression comprising COL4A3BP, MBNL2, FABP4, COL4A1, and UBE2C after 24 months. FIG. 6B shows the same gene signature after 36 months and FIG. 6C shows the signature after 60 months.

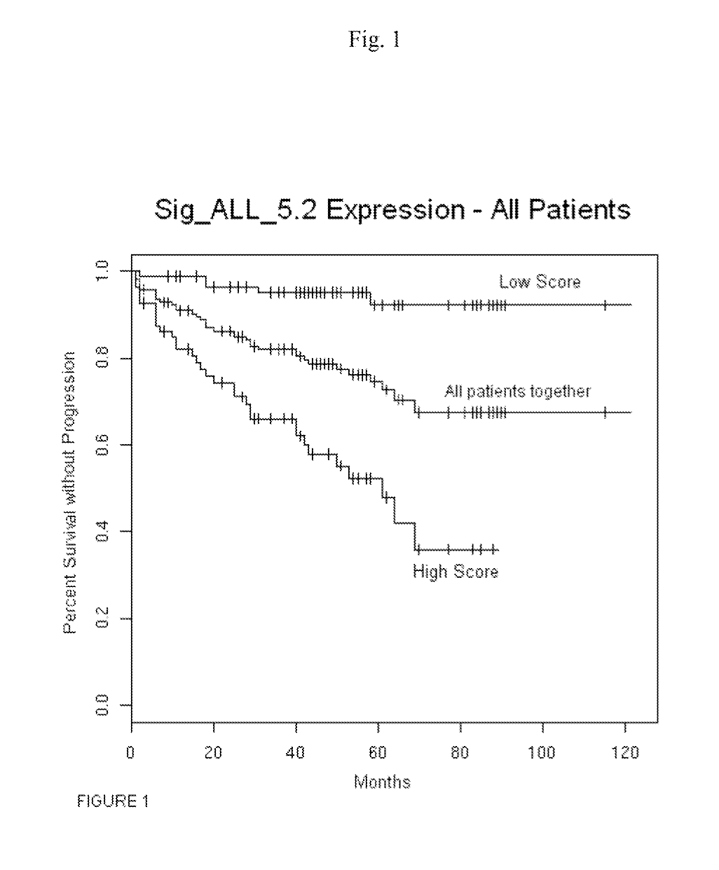

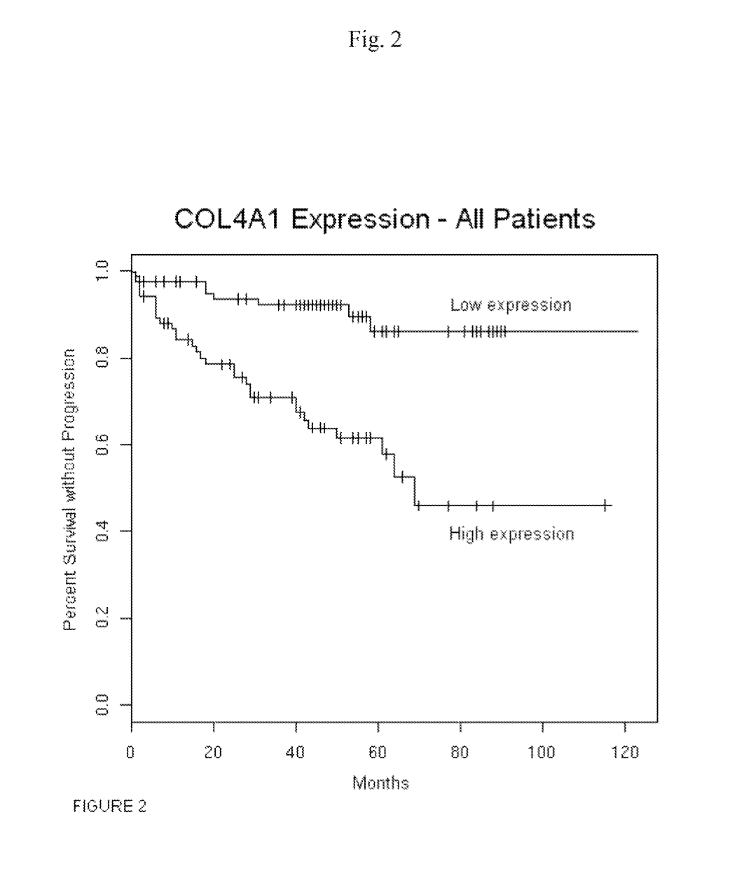

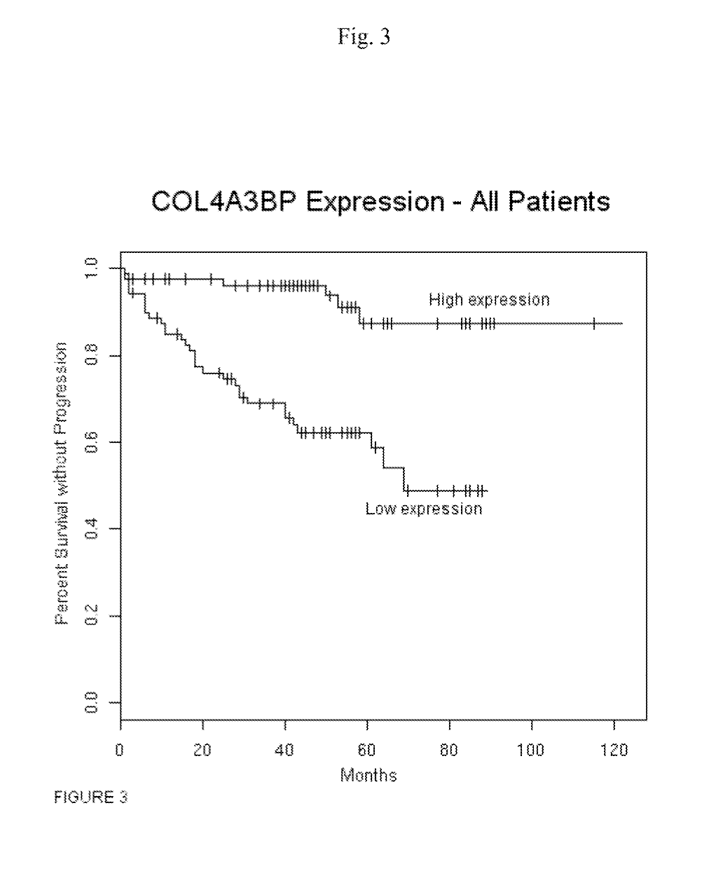

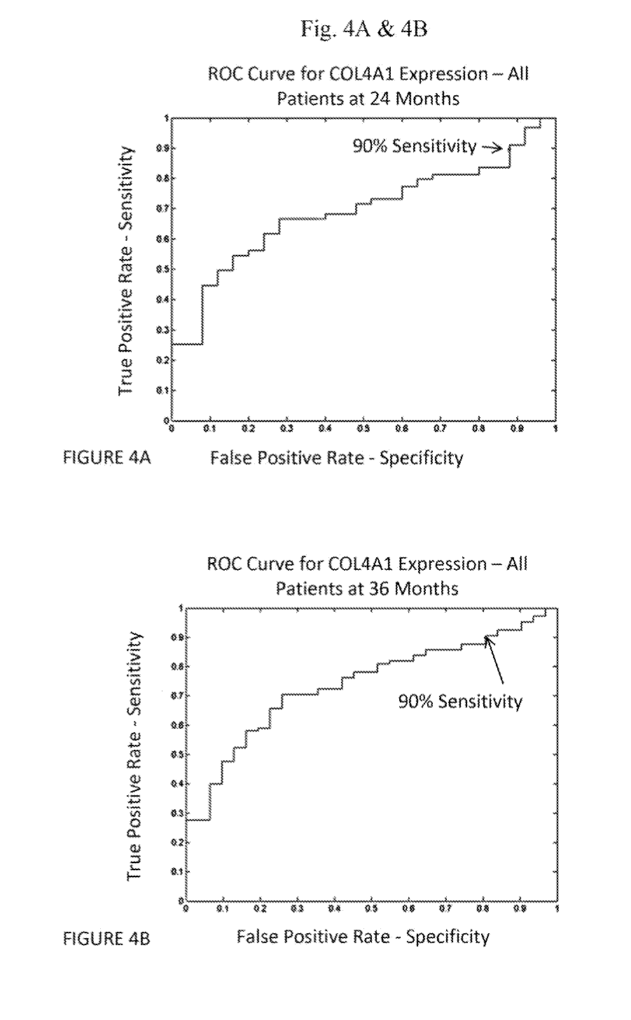

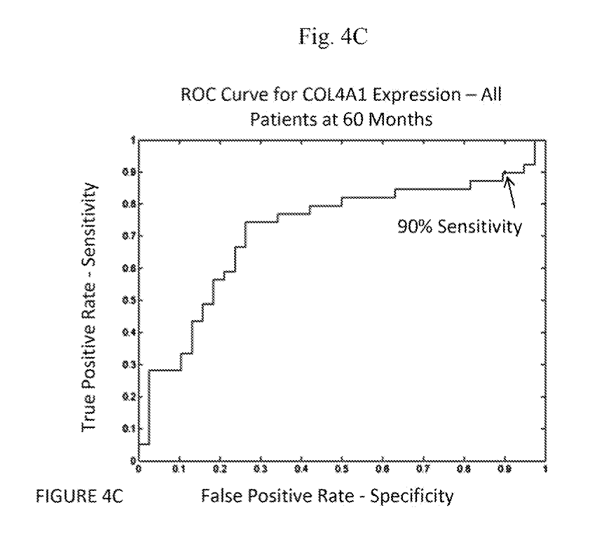

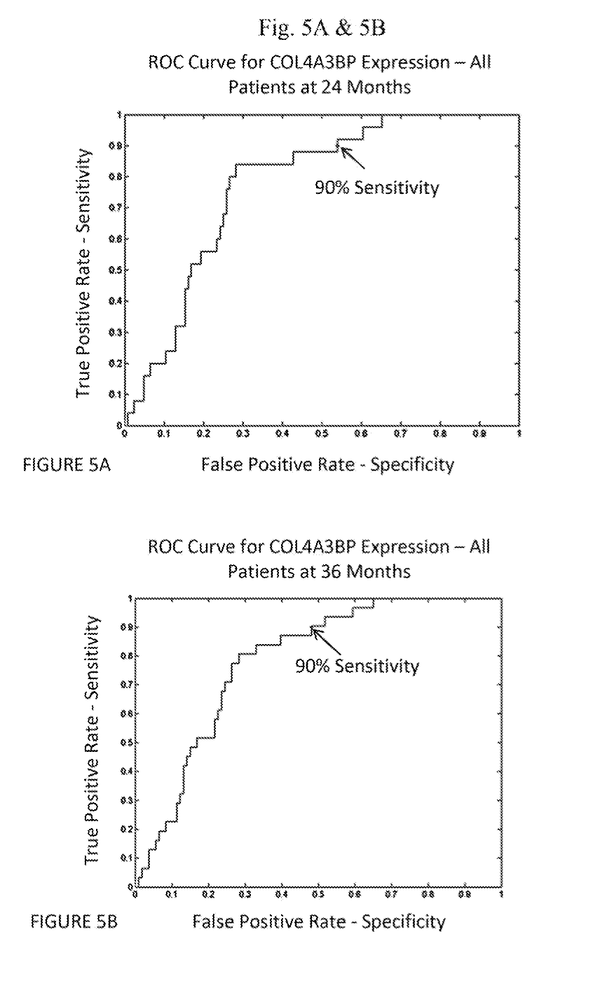

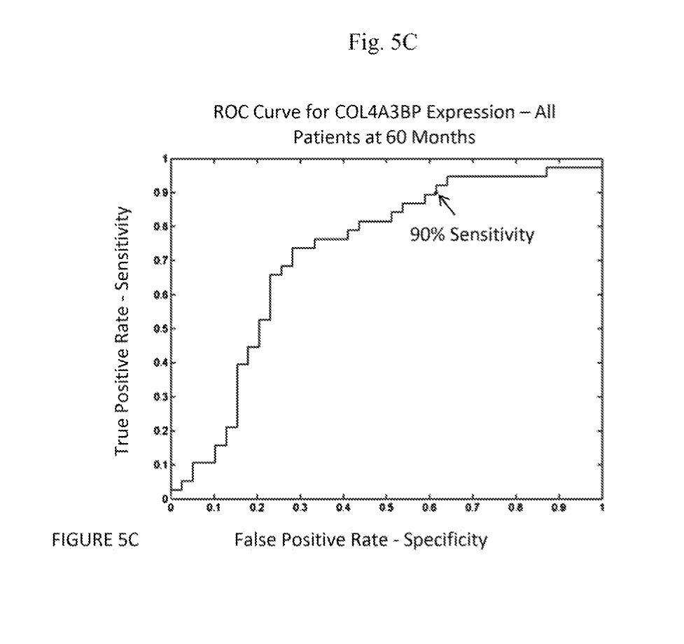

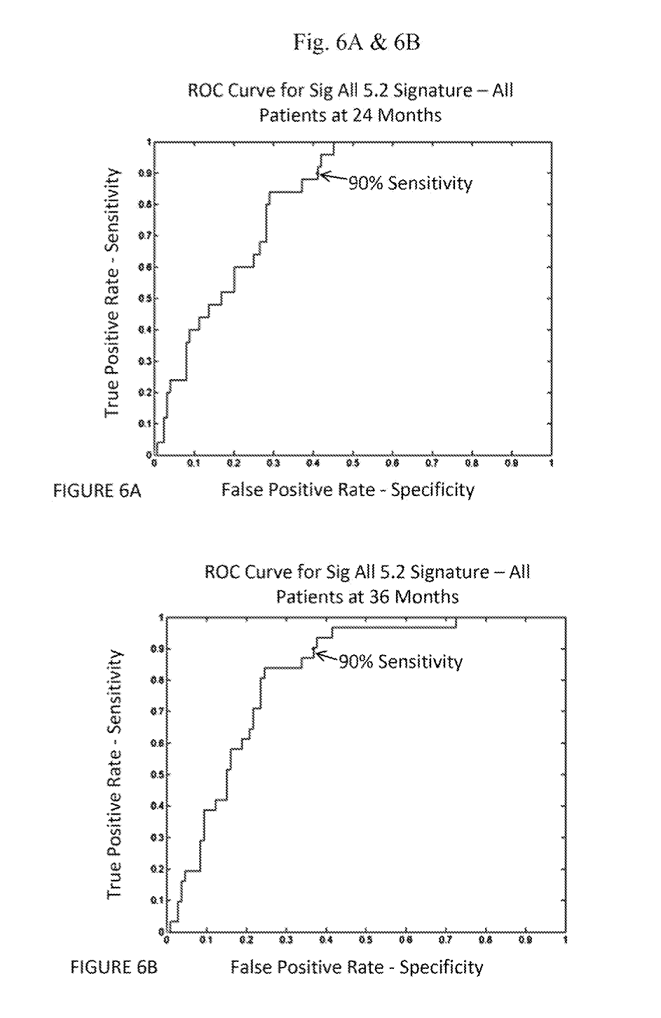

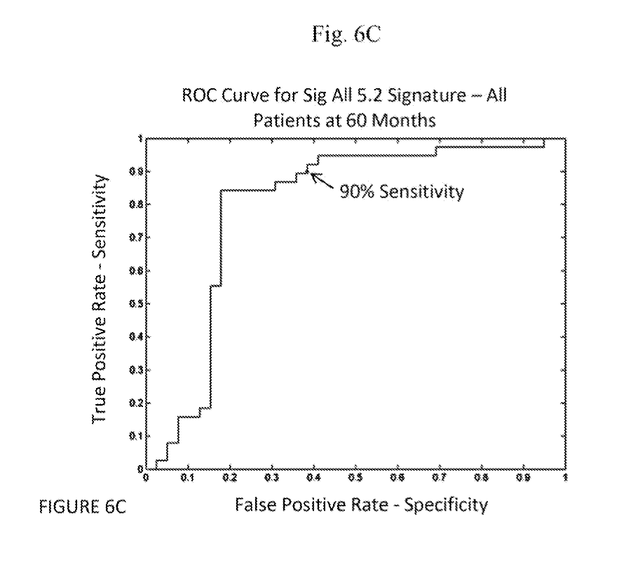

### SEQUENCE LISTING GUIDE

Sequences 1-92 and 94-108 in the sequence listing correspond to primer sequences (forward and reverse) and amplicon sequences immediately after each primer pair sequence for the 36 markers described in Example 3 below.

The sequences listed below correspond to one complete gene sequence of one isoform or transcript variant of the designated genes, following transcription processing as posted and available on the NCBI Nucleotide database.
SEQ ID NO. 93: SKAP2
SEQ ID NO. 109: COL4A1
SEQ ID NO. 110: NEK1
SEQ ID NO. 111: UBE2C
SEQ ID NO. 112: MBNL2
SEQ ID NO. 113: FABP4
SEQ ID NO. 114: BIRC5
SEQ ID NO. 115: COL18A1
SEQ ID NO. 116: ACTA2
SEQ ID NO. 117: MSN
SEQ ID NO. 118: KPNA2
SEQ ID NO. 119: COL4A3BP
SEQ ID NO. 120: CDC25B

### DETAILED DESCRIPTION

### Definitions

“Averaged value” for a signature, is the value obtained when the expression level of two or more genes or markers is averaged. Average value for a measurement, is the value obtained when two or more measurements of the same substance or marker are averaged. Typically the mean is used to compute the average value; although the median, mode, geometric average, or other mathematical average might be used.

“Concentration” when used as a noun refers to quantity(ies) of a substance(s) (such as a gene product) per unit of volume. Quantities can be measured or computed in units of mass, or molecules, or moles, or light absorption, or light emission, or radioactive emission, or other units that reflect the mass, number of molecules or moles of a substance. The phrase “per unit of volume” refers to a volume of tissue, cells, or fluid or other proxy for such volume the substance(s) was extracted from or measured in. For example, a concentration may be measured in molecules of a substance per gram of tissue (where the gram of tissue is a proxy for a volume of tissue weighing a gram); or micrograms of substance per cubic millimeter of tissue; or fluorescent light units emitted by a substance per microgram of total RNA extracted from a tissue (where a microgram of total RNA is a proxy for the volume of tissue that the substance and the microgram of total RNA were extracted from). If cells of a particular sample are relatively uniform and homogeneous, then the number of cells can be a proxy for cell volume.

“Control” refers to a bladder cancer sample or pool of bladder cancer samples that are used for comparison with a bladder cancer sample from a patient. In certain instances a control can be a normal non-cancerous sample.

“Ct score” refers to a mathematical combination of Ct values, typically treating the unfavorable marker Ct values as a group and the favorable marker Ct values as a group. Ct values for markers may be combined using various mathematical functions. For example, Ct score may involve computing the mean, median, or mode of certain Ct values; or may involve computing one or more ratios, products, sums, differences, logarithms, exponents, and/or other mathematical functions.

“Ct value” in quantitative RT-PCR, is the PCR cycle number in which amplicon signal for a gene product first exceeds a detection threshold. The mRNA copy number of an indicator gene is proportional to 2^{-Ct} for that indicator gene; thus, when the difference between the Ct of a first gene product and the Ct of a second gene product increases, the relative amount of the first gene product in relation to the second gene product has decreased. A lower Ct value indicates a greater concentration of a gene product in a sample.

“Cut-off score” refers to a score associated with a signature allowing classification of patients into different prognostic or treatment groups. There may be more than one cut-off score for a diagnostic or prognostic test. For example, a first, lower cut-off score may be useful to separate patients into groups appropriate for treatment options A versus B; and a second, higher cut-off score may be useful to separate patients into groups appropriate for treatment options B versus C. The cut-off score for a signature may be determined from or with reference to the relative expression levels or the standard expression levels for the gene products in the signature or by other means or from other references.

“Cut-off value” refers to an expression level of a gene product allowing classification of patients into different prognostic or treatment groups. There may be more than one cut-off value for a diagnostic or prognostic test. For example, a first, lower cut-off value may be useful to separate patients into groups appropriate for treatment options A versus B; and a second, higher cut-off value may be useful to separate patients into groups appropriate for treatment options B versus C. The cut-off value for any gene product may be determined from or with reference to the relative expression level or the standard expression level for that gene product, or by other means or from other reference.

“Expression” or “expressed” when referring to a gene product means a biological activity leading to the production of that gene product.

“Expression levels” or “level of gene expression” refers to quantity(ies) of gene product(s) per unit of measure, such as per cell, or per milliliter, or per cubic millimeter, or per gram of tissue. Expression levels or level of gene expression may be quantified as a concentration.

“Favorable markers” is used synonymously with protective markers.

“Gene” refers to a genomic sequence, including a marker sequence. Genes may be expressed at different levels in cells or not expressed at all. A “gene” may be part of a genomic DNA sequence that is transcribed into RNA molecules. Such RNA molecules may or may not be spliced into mRNA and/or translated into protein. Gene as used herein may be any part or several parts of a genomic DNA sequence that may be transcribed into RNA molecules. The genes/markers COL4A3BP, MBNL2, FABP4, NEK1, COL4A1, UBE2C, BIRC5, COL18A1, KPNA2, MSN, ACTA2, SKAP2, and CDC25B are designations for these genes as referenced in the US National Institutes of Health National Center for Biotechnology Information (NCBI) database and publically available since the earliest priority date of this application, and the sequences corresponding to each of the genes in the Sequence Listing Guide above are the complete sequence of one isoform of the designated genes following transcription processing and thus, can be used in determination of the quantity of a particular expression product.

“Gene expression” refers to a biological activity measured by the level or concentration of one or more gene products in a sample.

“Gene product” refers to molecules(s) that are derived directly or indirectly from genomic DNA (including from genes or markers therein) as a result of gene expression in vivo, or from derivatives of such in vivo gene expression created in vitro. Examples include RNA, miRNA, pre-mRNA, mRNA, cDNA copied from RNA, nucleic acid copies or amplification products derived from the previous nucleic acid forms, and protein translated from the previous nucleic acid forms.

“Harmful markers” are indicator genes or indicator gene products for which increased expression levels indicate a less favorable prognosis, i.e., increased expression levels correlate with higher risk of progression; and decreased expression levels correlate with lower risk of progression.

“Increase (or decrease) in risk of progression” refers to a relative increase or decrease in likelihood of progression. If expression levels of several markers are determined in succession, the relative likelihood of progression will increase or decrease as the expression level of each successive marker is determined and analyzed.

“Indicator genes” or “indicator gene products” are genes or gene products that are useful for disease prognosis, particularly bladder cancer progression and prognosis, and comprise both favorable and unfavorable genes or gene products. Examples include, but are not limited to COL4A3BP and COL4A1.

“Individual” as related to the source of a bladder cancer sample, refers to an animal, preferably a human.

“mRNA” refers to RNA molecules that are produced from genes, typically through pre-mRNA intermediates. mRNA may be translated into protein. mRNA may be reverse-transcribed into cDNA.

“Marker” is used synonymously with indicator gene or indicator gene product.

“Non-progression” (or “non-progressors’) in reference to bladder cancer or bladder cancer patients refers to lack of progression from either bladder cancer stage Ta or T1 to: (i) any of the more advanced stages T2 through T4, or (ii) death from bladder cancer.

“Progression” (or “progressors”) in reference to bladder cancer or bladder cancer patients refers to progression from either bladder cancer stage Ta or T1, to: (i) any of stages T2 through T4, or (ii) death from bladder cancer. “Progression” (or “progressors”) in reference to bladder cancer or bladder cancer patients may also be defined as (i) invasion into the bladder muscle; (ii) more distant metastases; or (iii) death from bladder cancer with or without verified progression.

“Progression-free survival” is the time between the first diagnosis or resection of the analyzed bladder tumor and, either identified progression or the patient dropping out of the study (preventing taking of results) without progression.

“Protective markers” are indicator genes or indicator gene products for which increased expression levels indicate a more favorable prognosis, i.e., increased expression levels correlate with non-progression; and decreased expression levels correlate with risk of progression.

“Quantity(ies)” in reference to expression levels refers to the concentration or copy number of a gene product.

“Relative quantity(ies)” refers to the amount, concentration, or copy number of one or more gene products relative to the amount, concentration, or copy number of one or more other gene products, particularly other markers.

“Relative expression level” refers to the expression level of one or more gene product(s) as compared to a) a known expression level of the same gene product(s) and/or b) a known or unknown but measured expression level of another gene product(s). Other gene products may be a single gene product or a collection of gene products, or a cell's or tissue's gene products.

“Score” refers to the result of a mathematical computation using one or more marker expression levels, typically treating the unfavorable marker level(s) as a group and the favorable marker level(s) as a group. Expression levels for markers may be combined using various mathematical functions. For example, determining score may involve computing the mean, median, or mode of certain expression levels; or involve computing one or more ratios, products, sums, differences, logarithms, exponents, and/or other mathematical functions. It is contemplated that in some cases only one gene or marker will be present in a group for which a score is determined.

“Signature” refers to sets or groups of markers.

“Standard expression level” refers to the expression level of one or more gene product(s) in a standard situation such as an expression level associated with non-progression of bladder cancer or an expression level associated with progression of bladder cancer.

“Unfavorable markers” is used synonymously with harmful markers.

### DETAILED DESCRIPTION

### Treatment

Bladder cancer patients are typically monitored with cystoscopy at intervals that vary from four to twelve months. Recurrence is very common in bladder cancer and therefore frequent monitoring with cystoscopy is required for all patients. The most aggressive cases of bladder cancer with high stage and grade are currently monitored with the shortest intervals as patients with aggressive bladder cancer have the most frequent recurrences. The high number of required cystoscopies makes bladder cancer one of the most costly cancers to treat on a per capita basis. Estimates of bladder cancer aggressiveness are determined from the bladder cancer patient's disease course history in the previous one to two years and would benefit greatly from biomarkers that could inform the doctor of aggressiveness at each patient visit. Bladder cancer disease state can change from indolent to aggressive during the course of the disease.

Bladder cancer treatment is currently determined by how deeply the tumor invades into the bladder wall and how frequently recurrences occur. Superficial tumors (those not entering the muscle layer) can be removed using an electrocautery device attached to a cystoscope, which in that case is called a resectoscope. The procedure is called transurethral resection (TUR) and serves primarily for pathological staging and tumor removal. In case of non-muscle invasive bladder cancer, the TUR is itself the treatment, but in case of muscle invasive cancer, the procedure is insufficient for final treatment (see below). In cases with frequent recurrences, immunotherapy in the form of BCG (bacillus Calmette-Guerin) instillation is also used to treat and prevent the recurrence of superficial tumors, and may also prevent up-staging in aggressive cases.

BCG immunotherapy is effective in the majority of superficial tumors, especially in high-risk patients with extended pre-malignant alterations in the bladder; the so-called “field defect.” Instillations of chemotherapy, including use of valrubicin (Valstar) into the bladder can also be used to treat BCG-refractory CIS disease when cystectomy is not an option. Both treatments are also used in combination with resection of the bladder tumor.

Patients whose tumors recur after treatment with BCG are more difficult to treat. Many physicians (and the official guidelines of the European Association of Urologists (EAU) and the American Urological Association (AUA)) recommend cystectomy for these patients. In cystectomy, part or all of the bladder is removed and the urinary stream is diverted into an isolated bowel loop (called an ileal conduit or urostomy). In some cases, skilled surgeons can create a substitute bladder (a neobladder) from a segment of intestinal tissue. Many patients prefer to try other treatment options first before undergoing this life changing operation.

Device assisted chemotherapy is one such group of novel technologies used to treat superficial bladder cancer. These technologies use different mechanisms to facilitate the absorption and action of a chemotherapy drug instilled directly into the bladder. Another technology uses an electrical current to enhance drug absorption. Still another technology, thermotherapy, uses radio-frequency energy to directly heat the bladder wall, which together with chemotherapy shows a synergistic effect, enhancing the capacity of each treatment to kill tumor cells.

Untreated, superficial tumors may gradually begin to infiltrate the muscular wall of the bladder. Tumors that infiltrate the bladder wall usually require cystectomy. A combination of radiation and chemotherapy can also be used to treat invasive disease. Chemotherapy may also be used before cystectomy. Unfortunately, only approximately 50% of patients respond to chemotherapy so cystectomy is frequently still required.

In the preferred embodiment of the invention, treatment is determined based on the expression levels of the harmful and protective markers. In cases where progression is likely or risk of progression is increased compared to control(s), based on such expression levels, the treatment regimen could include BCG instillation(s), and optionally, also chemotherapy or cystectomy.

Expression Level Determination

For measuring the amount of a particular RNA in a sample, various well-known methods can be used to extract and quantify transcribed RNA from a patient sample. Briefly, RNA can be extracted from a sample such as a tissue, body fluid, or culture medium in which a population of cells of a subject might be growing. For example, cells may be lysed and RNA eluted in a suitable solution in which to conduct a DNase reaction. First strand synthesis may be performed using a reverse transcriptase enzyme. Gene amplification, more specifically quantitative PCR (QPCR) assays, can then be conducted. Samples are preferably run in multiple replicates, for example, 3 replicates.

In an embodiment of the invention, QPCR is performed using dNTPs, primers, buffer and polymerase enzymes suitable for QPCR, reporting agents such as intercalating dyes, minor groove binding dyes, labeled probes or other such agents known in the art, and instruments, including those supplied commercially by Applied Biosystems (Foster City, Calif.). Ct value or other quantifiable signals such as fluorescence, enzyme activity, disintegrations per minute, absorbance, etc., when correlated to a known concentration of target templates (e.g., a reference standard curve) or normalized to a standard, can be used to quantify the mRNA quantity in an unknown sample.

One embodiment of the invention is an assay for determining increased and decreased expression of harmful markers and protective markers, and methods of analyzing the results from such an assay. The increased or decreased expression is determined relative to the expression level(s) of the harmful and protective markers in progressors and/or in non-progressors, as described in further detail below.

In one embodiment, the assay results for particular harmful or protective markers can be used individually or in signatures to determine the likelihood of progression or non-progression. The assay results are used to form signatures comprising the markers found to be most significant in predicting the likelihood of bladder cancer progression or non-progression through a variety of statistical tests. One important calculation determined for a signature is a score. Scores are used in predicting the likelihood of bladder cancer progression or non-progression.

In one embodiment, finding the difference between the averaged values for the unfavorable markers and the averaged values for the favorable markers for a particular signature used in determining a score may reduce the noise and provide more reliable assay results. The same method of noise reduction can be applied to other types of assays where values representing markers of interest are combined into signatures, including assays not involving harmful or protective markers, as well as assays not measuring Ct values or determining average Ct values or Ct scores. Use of the method in any such application is also within the scope of the invention.

Another embodiment relates to using the determination of the increased and decreased expression of harmful markers and protective markers, or scores, in tailoring the patient's treatment. For example, a more aggressive treatment may be indicated in a stage Ta or T1 patient where the harmful markers are increased and/or the protective markers are decreased, as such a patient would be more likely to undergo progression to a more advanced stage of bladder cancer. Non-limiting examples of more aggressive treatments may include larger dosages of chemotherapy, different or additional chemotherapy agents, combinations of chemotherapy with other therapies (including radiation therapy), or surgery. It will be obvious to one of skill in the art that as new treatments are developed these could also be used in individuals at risk of progressing to a more advanced stage of bladder cancer. If one assumes that aggressive medical/surgical measures present higher morbidity-mortality-risks/discomforts/reduced quality of life/costs for the patients, then assessing risk of progression is highly useful in making these difficult decisions on course of treatment. In any event, the likelihood of progression or non-progression as determined by the methods herein is useful and important information for the patient and his/her health care team in making a variety of care, treatment and lifestyle choices.

In a first embodiment, the invention provides a method for predicting likelihood of a patient's progressing from stage Ta or T1 bladder cancer to a more advanced stage. This method comprises: (a) obtaining tissues or samples from a number of patients with stage Ta or T1 bladder cancer; (b) monitoring the patients for a sufficient period such that there are enough progressors and non-progressors among the patients to provide statistically significant results from the indicator gene or indicator gene product monitoring; (c) comparing quantities of certain indicator genes or indicator gene products between the progressors and the non-progressors to determine which such genes or gene products are useful markers, and among the indicator genes or indicator gene products: (i) which are favorable markers, i.e., which of the indicator genes or indicator gene products have a statistically significant association between increases in their quantities and non-progression, and/or decreases in their quantities and progression; and (ii) which are unfavorable markers, i.e., which of the indicator genes or indicator gene products have a statistically significant association between increases in their quantities and progression and/or decreases in their quantities and non-progression; (d) establishing a cut-off value for each or a group of the indicator genes or indicator gene products, and (e) wherein a patient's expression level of one or more indicator genes or indicator gene products is compared with the cut-off values for such indicator genes or indicator gene products to determine the likelihood of the patient being a progressor or a non-progressor.

In a second embodiment, following steps (a), (b) and (c) above in the first embodiment, one performs the following steps: (d) forming signatures and determining the signatures having scores which indicate progression and/or non-progression with statistical significance; (e) determining a cut-off score for each signature, wherein (f) a patient's gene signature(s) is compared with the cut-off score for that signature to determine the likelihood of the patient being a progressor or a non-progressor.

In one preferred embodiment, QRT-PCR is used to assay quantities of indicator gene products, and the Ct value obtained from the assay correlates with the quantities of indicator gene products. The cut-off value is determined from Ct values of progressors and non-progressors (using the method of cut-off value determination described in the first embodiment) the cut-off score is determined from Ct scores, and a patient's Ct score is compared to the cut-off score (as described in the second embodiment).

In either the second embodiment or the preferred embodiment above, the determination of the score or Ct score may reduce the noise and provide more accurate results. This noise reduction method can be applied in certain other types of assays, including assays where signals representing gene expression levels are other than Ct values; for example, a fluorescent signal emitted by amplicons from cDNA templates of the markers, or a signal generated from a microarray corresponding to the gene expression levels, or a signal generated from measurement of protein levels translated from the mRNAs which were expressed. All such noise reduction methods are further embodiments on the invention.

In any of the foregoing embodiments, the cutoff value would represent a quantity of mRNA or marker somewhere between the extreme low and extreme high relative quantities observed in progressors and non-progressors. Often the cutoff value will represent the average, mean or median quantity of indicator genes or indicator gene products found in a collection of specimens derived from groups of bladder cancer patients having relatively equal proportions of progressors and non-progressors. However, it may be advantageous to use a cutoff value that is different from the average, mean or median amount, especially in situations where false positive or false negative test results have unequal clinical implications. That is, one may want to set the cut-off value to reduce false negatives at the expense of increasing false positives, or vice-versa. The cut-off value can also be a value representing a recognized standard quantity of a marker, associated with a progressor or non-progressor; or it can be based on but different from such standard-based value; again, where false positive or false negative test results have unequal clinical implications. The cut-off score is determined under essentially the same considerations as the cut-off value, but it is calculated using scores from signatures of progressors and non-progressors, rather than from measures of mRNA or marker quantities.

Genes and Primer Sequences

In the experiments described below, the QRT-PCR used PCR primers that hybridized to regions of the mRNA that were located relatively close to each other on the mRNA molecule, making relatively small amplicons. Small amplicons will typically amplify more efficiently than large amplicons and for this reason amplicon sizes between 50 and 150 bp are preferred and amplicons between 60 and 95 bp are particularly preferred. The sequences of the QRT-PCR amplification primers (forward and reverse) are set forth in Table 1. The designations of the indicator gene products and other markers and genes referenced herein are as provided on NCBI, available on the internet. Quantities of the designated markers can be determined by amplification with the primers in Table 1, or any other primer or probe which amplifies (or hybridizes to) any portion of these markers, including any mutant or polymorphic forms, and transcript variants.

The primer set selected should amplify the mRNA loci which is transcribed from the marker, and preferably should minimize amplifying, or generating signal from, genomic DNA or transcripts or mRNA from related but biologically irrelevant loci other than the target loci. A number of different primer sets can be selected under this criterion to amplify mRNA transcribed from the indicator genes of interest.

One can also monitor expression of these markers by monitoring the protein expressed from them, using techniques for assaying for particular protein products, including ELISA, Western Blotting, and enzyme assays.

TABLE 1
Primer Sequences, Sensitivity, Specificity, and Optimal Concentration
for 13 Preferred Genes/Markers

| | | | | | Primer Pair | Primer Pair |
|---|---|---|---|---|---|---|
| Gene | Forward Primer | Optimal | Reverse Primer | Optimal | Sensitivity | Specificity |
| Name | Sequence 5′-3′ | [nM] | Sequence 5′-3′ | [nM] | (ng) | (NTC Ct) |
| ACTA2 | GTCTCTAGCACAC | 200 | CTAGGAATGATTT | 200 | 0.1 | No Ct |
| | AACTGTGAATGTC | | GGAAAAGAACTG | | | |
| BIRC5 | CTGAAGTCTGGCG | 200 | GAAGCTGTAACAA | 200 | 0.1 | No Ct |
| | TAAGATGATG | | TCCACCCTG | | | |
| CDC25B | GATGGAAGGTTGG | 200 | ACCTGGTTTGGGT | 200 | 0.01 | No Ct |
| | ATGGATG | | ATGCAAG | | | |
| COL18A1 | GGGCTGGTTCTGT | 200 | AAAAGGTCACTTT | 200 | 0.01 | No Ct |
| | AATTGTGTG | | TATTTGCCTGTC | | | |
| COL4A1 | CTGCCTGGAGGAG | 200 | CTGTAAGCGTTTG | 200 | 0.1 | No Ct |
| | TTTAGAAGTG | | CGTAGTAATTG | | | |
| COL4A3BP | TTTCTGTGGATCA | 200 | CAAGGTTTGACAA | 200 | 0.1 | No Ct |
| | TGACAGTGC | | ATCATAGCAAC | | | |
| FABP4 | AGAGAAAACGAGA | 200 | CTTATGCTCTCTCA | 200 | 0.01 | No Ct |
| | GGATGATAAACTG | | TAAACTCTCGTG | | | |
| KPNA2 | GCAGATTTTAAGA | 300 | AAGGTACACAATCT | 100 | 0.1 | No Ct |
| | CACAAAAGGAAG | | GTTCAACTGTTC | | | |
| MBNL2 | ACTTCATCCAGTG | 50 | GGGGTTACAGGTGC | 350 | 1.0 | >40 |
| | CCCACTTTC | | TAGGTAAGG | | | |
| MSN | CCTGACCTTGAGG | 200 | AATATAGGACATAT | 200 | 0.1 | No Ct |
| | TAGCTTGTG | | CACCAAGTGAGC | | | |
| NEK1 | CTAAAAGACCAGC | 200 | CTAAAGGTATTCCA | 200 | 0.1 | No Ct |
| | CTTAGGACAAAAC | | TTATTAGCGGC | | | |
| SKAP2 | TGGAGATGTATGA | 200 | CTAAATCCAAAGCA | 200 | 0.1 | No Ct |
| | TATTTGAGAGTCC | | TTTGCAGAC | | | |
| UBE2C | TCTAGGAGAACCC | 200 | TCTTGCAGGTACTT | 200 | 0.01 | No Ct |
| | AACATTGATAGTC | | CTTAAAAGCTG | | | |

Experimental Determination of Favorable and Unfavorable Markers

### EXAMPLE 1

### Patients and Biological Material

The favorable and unfavorable markers described herein were found by analysis of samples from a study of 205 patients presenting with Ta or T1 stage bladder cancer (8 patients presented with stage T2 bladder cancer and were removed from much of the subsequent data analysis). The tumor samples were taken from patients that were operated on between 1987 and 2000 in hospitals in Denmark, Sweden, Spain and England. Biological materials were obtained directly from surgery after removal of the necessary amount of tissue for routine pathology examination. Informed written consent was obtained from all patients and research protocols were approved by the institutional review boards or ethical committees in all involved countries. Diagnostic pathology slides were evaluated according to the WHO guidelines. The patients were studied for a minimum of 60 months, with gene expression analysis at baseline, and with patients followed for non-progression or progression (including death from bladder cancer) at several regular intervals. Progression free survival time was recorded from the sampling visit and censored at the time of the last control cystoscopy or at cystectomy. If a patient died of bladder cancer, survival was recorded from the sampling visit until the last annotation of the patient being alive.

Patients with stage T1, or patients with stage Ta but also with carcinoma in situ (CIS), or patients with high grade (including the small group of stage T2 patients) were classified by clinicians as “high risk” (a group of 131 patients). Patients with stage Ta without CIS and low grade were classified by clinicians as “low risk” (74 patients). QRT-PCR, as described below, was used to analyze the patients' mRNA. The patient mRNA for the study was purified from patient bladder cancer tissue biopsied in routine cystoscopy.

TABLE A
Patient Sample Clinical Characteristics
Clinical Characteristics for all 197 Patients with Ta or T1 Cancer

| | |
|---|---|
| Number of Patients | 197 |
| Median follow-up time in months for all patients (range) | 40 (0-170) |
| Median follow-up time in months for progressing patients | 28 (0-170) |
| (range) | |
| Median follow-up time in months for non-progressing | 42 (0-115) |
| patients (range) | |
| Median age (range) | 72 (27-94) |
| Male-female ratio | 4.3 |
| Stage | |
| Ta | 106 |
| T1 | 91 |
| Grading (WHO 2004) | |
| PUNLMP | 28 |
| Low Grade | 51 |
| High Grade | 118 |
| Concomitant CIS | |
| Yes | 30 |
| No | 58 |
| Unspecified | 109 |
| Adjuvant Therapy (BCG or MMC) | |
| Yes | 48 |
| No | 149 |
| Number of Progression events to stage T2-4 bladder cancer | |
| Ta | 11 |
| T1 | 26 |

### EXAMPLE 2

### RNA Extraction and cDNA Generation

Total RNA was extracted from the biopsied bladder tumor samples using a standard Trizol RNA extraction protocol (Invitrogen) in the case of the Danish and English samples or using the RNeasy mini kit (Qiagen) for the Swedish and Spanish samples. Quality of the extracted RNA was verified using an Agilent Bioanalyzer where 28S/18S>1 and RIN>5 were the criteria used. Then the total RNA was DNase treated using amplification grade DNase I (Invitrogen) to degrade any genomic DNA present in the purified total RNA. To verify the complete digestion of any contaminating genomic DNA, the RNA sample was amplified in a QRT-PCR reaction with GAPDH primers that hybridized to GAPDH at intron-exon junctions and thus could only hybridize to and amplify genomic DNA. The DNase treated total RNA was converted to cDNA using oligo (dT) priming and SuperScript II Reverse Transcriptase (Invitrogen) under standard protocols.

### EXAMPLE 3

### Selection of Genes/Markers for Analysis

To identify and validate our bladder cancer progression markers we started with 36 markers that looked promising as progression indicators in an earlier validation study with a different population of 101 patients. These 36 markers were identified using microarrays and verified with QPCR. The 36 markers were selected based upon the separation of individuals with higher expression and lower expression of each marker in Kaplan Meier survival plots as well as looking at t-test results. In the current study these markers were re-tested using QRT-PCR with 384 well plates and samples from the 205 patients. For each patient, 3 replicate reactions were quantified for each of the 36 markers of interest, and the results from the 3 reactions were averaged.

For some computations, especially when individual marker levels were being studied, Ct values were normalized across the entire population of patients. However, non-normalized signals were generally used in other determinations, especially when signals of groups of markers were combined into signatures.

The values normalized across the patient population were used to find markers for associations between Ct values (representing quantities of markers from gene expression) and clinical events; i.e., disease progression or bladder-cancer-related death (it was assumed that death from bladder cancer involved progression of the disease stage, even if this progression had not been detected prior to death) versus non-progression. Analysis of these results led to selection of markers of interest, which appeared to have high or low expression levels that correlated well with the clinical determinations of either progression (including death from bladder cancer) or non-progression.

To determine the unfavorable markers associated with progression/death, the statistical correlations between mRNA expression levels and progression of disease were studied. Movement from stage Ta to stage T1 was not deemed progression.

### EXAMPLE 4

### Primer Design and PCR Assays

Three primer pairs were designed for each marker selected, using Primer3 free software. A pool of total RNA from 14 bladder tumors of different stages and grades was used for cDNA synthesis as described above to use for testing the sensitivity and specificity of the primer designs. Input cDNA in the reactions was 1 ng, 0.1 ng, and 0.01 ng and a minimum of two replicates had to amplify at a particular input cDNA concentration to qualify as the limit of sensitivity. Therefore, if two or more replicates amplified with 0.01 ng cDNA for a particular primer pair, the sensitivity of that primer pair was 0.01 ng. The Ct values for each replicate had to be less than one cycle apart to meet the sensitivity requirements. To determine specificity, no template control reactions were examined for Ct values before 40 cycles. If this occurred, the primer pair did not pass the specificity requirements. The primer pairs selected for preferred markers are shown in Table 1 along with details about optimal primer concentration and primer specificity and sensitivity as described in this example. Then QRT-PCR was performed on a 7900HT Fast Real-Time PCR System (Applied Biosystems) in 384 well plates. All reactions were performed in triplicate in 10 μL volumes using SYBR Green PCR Master Mix (Applied Biosystems) under standard protocols.

### EXAMPLE 5

### Analysis, Gene Selection and Signature Identification

After determining the markers of interest, sets of markers (signatures) were identified and studied. More specifically, the procedure followed for finding the signatures was:

1) for each marker, the Pearson correlation coefficient between averaged triplicate Ct results and the clinical parameter (e.g. progression including bladder-cancer-related death) was calculated where P<0.01;

2) using the average of the triplicate values of the Ct results for each marker, a t-test, Wilcoxon signed-rank test, P<0.01, Kolmogorov-Smirnov (KS) test, P<0.01, and Chi-squared test, P<0.01, were run to evaluate statistical differences in gene expression in different subpopulations of patients (e.g. progression including bladder-cancer-related death vs. no progression or bladder-cancer-related death);

3) using the average of the triplicate values of the Ct results for each marker, some additional analyses, i.e. Kaplan-Meier plots (measuring progression-free survival), receiver operating characteristic curves (ROC), AUC>0.65, and Cox regression analyses were performed, P<0.01;

4) the markers that performed the best in all or most of the above criteria 1-3 were defined and separated into several groups, based on whether higher relative expression levels were correlated with progression or non-progression.

In order to increase the signal to noise ratio in each signature, without using the mRNA expression values normalized across the patient population (as they were observed to lead to less correlative results), the average of the triplicate values of the Ct results for each marker was calculated and then all such averaged Ct values for the unfavorable markers were subtracted from averaged values for the favorable markers for each signature studied. By subtracting the averaged Ct value of one set of markers from the averaged Ct value of another set of markers, one is subtracting out much of the noise. That is, if a variable (such as mRNA preparation methods) affects the measurement of favorable markers, the same variable will likely also affect the measurement of unfavorable markers.

This method of increasing the signal to noise ratio where one forms signatures representing gene expression levels could also be used where signals representing these levels are other than Ct values; for example, a fluorescent signal emitted by amplicons from cDNA templates of the markers, or a signal generated from a microarray corresponding to the gene expression levels, or a signal generated from measurement of protein levels.

When the average value computed for the unfavorable markers is subtracted from the average value computed for the favorable markers, the difference is the score.

For each signature, comparing the score with a cut-off score determines likelihood of progression or non-progression. In this example, the score is based on Ct values. A score with higher expression of favorable markers than of unfavorable markers relative to a cut-off score indicates a likelihood of non-progression. Inversely, a signature with a score showing higher expression of harmful markers than of protective markers relative to a cut-off score indicates a likelihood of progression.

Patients were classified into two groups for each signature examined: (i) below cut-off score and (ii) above cut-off score. Then the Chi square test was applied to determine the independence from progression or death from bladder cancer at 24, 36 and 60 months, for each patient. These time points were selected because they were relevant to patient therapy, however, any other time points for which clinical data was collected could have been selected. The expected value used in the test was that half of the patients (102) would fall into the below cut-off score group, and the other half (102) of the patients would fall into the above cut-off score group. P-values were calculated for each signature examined at 24, 36 and 60 months (again, other time points could have been used).

TABLE 2
A preferred predictive signature for determining the likelihood of progression and non-
progression. The number of patients is shown for each time point and in each clinical category.
The p-values for the Chi Squared test are also shown.
Each signature of interest was also analyzed using a t-test, Wilcoxon signed-rank test,
Kolmogorov-Smirnov test, Cox regression analysis, ROC curve, and Kaplan-Meier plot (for
progression-free survival), to evaluate statistical differences in marker expression in different
subpopulations of patients (e.g. progression including death from bladder cancer vs. non-
progression).

| | 24 MONTHS | | | | 36 MONTHS | | | | 60 MONTHS | | | |
|---|---|---|---|---|---|---|---|---|---|---|---|---|
| SIGNATURE | | | | Chi- | | | | Chi- | | | | Chi- |
| Sig All | Low | High | | squared | Low | High | | squared | Low | High | | squared |
| 5.2 | Score^{a} | Score^{b} | Total^{c} | p-value | Score^{a} | Score^{b} | Total^{d} | p-value | Score^{a} | Score^{b} | Total^{d} | p-value |
| No | 46 | 78 | 124 | 0.0048 | 36 | 70 | 106 | 0.0011 | 12 | 27 | 39 | 0.0177 |
| Progression | | | | | | | | | | | | |
| Progression | 16 | 3 | 19 | 0.0027 | 21 | 4 | 25 | 0.0006 | 27 | 5 | 32 | 0.0001 |
| Died of | 5 | 0 | 5 | 0.0246 | 5 | 0 | 5 | 0.0246 | 5 | 0 | 5 | 0.0246 |
| Bladder | | | | | | | | | | | | |
| Cancer | | | | | | | | | | | | |
| without | | | | | | | | | | | | |
| Progression | | | | | | | | | | | | |
| Detected^{d} | | | | | | | | | | | | |
| Progressed | 21 | 3 | 24 | 0.0002 | 26 | 4 | 30 | 0.0001 | 32 | 5 | 37 | 0.00001 |
| Or Died Of | | | | | | | | | | | | |
| Bladder | | | | | | | | | | | | |
| Cancer | | | | | | | | | | | | |
| Total | 67 | 81 | 148 | | 62 | 74 | 136 | | 44 | 32 | 76 | |
| Patients | | | | | | | | | | | | |
| Lost From | 34 | 21 | 55 | | 39 | 28 | 67 | | 57 | 70 | 127 | |
| The Study | | | | | | | | | | | | |
| CORRECTED | 101 | 102 | 203 | | 101 | 102 | 203 | | 101 | 102 | 203 | |
| TOTAL | | | | | | | | | | | | |
| (total + lost)^{e} | | | | | | | | | | | | |

^{a}A low score means that the average of all Ct's for the harmful markers was higher than the average of all Ct's for the protective markers in the signature and thus the likelihood of progression from bladder cancer was decreased because the protective markers have lower Ct's and are expressed at higher levels than the harmful markers.
^{b}A high score means that the average of all Ct's for the protective markers was higher than the average of all Ct's for the harmful markers in the signature and thus the likelihood of progression from bladder cancer was increased because a high Ct means low expression.
^{c}Total shows the information for all patients, both patients with low and high scores.
^{d}The patients in this category are known to have died from bladder cancer and therefore they must have progressed, however, the progression was not recorded in the patient's clinical record so they are categorized as a bladder cancer death without detected progression.
^{e}This total is 203 patients because a certain number of samples for the gene signature dropped out due to reaction failure or other reasons.

A preferred 5-marker signature determined to be very strongly correlated with clinical data and thus highly predictive of likelihood of progression or non-progression is shown in Table 3.

TABLE 3
Markers included in a preferred predictive signature (Sig All 5.2)
for determining the likelihood of bladder cancer progression or
non-progression.

| Marker | Type |
|---|---|
| COL4A3BP | Protective |
| FABP4 | Protective |
| MBNL2 | Protective |
| COL4A1 | Harmful |
| UBE2C | Harmful |

TABLE 4
Markers selected for their correlation with clinical
determination of bladder cancer progression
or non-progression and associated statistics.

| | | | Wilcoxon | | Cox Regression | | |
|---|---|---|---|---|---|---|---|
| | Type | T | signed- | | Analysis | | |
| | (P or | Test | rank test | KS Test | Beta | | ROC |
| Marker | H)* | P-value | P-value | P-value | Coefficient | P-value | AUC |

24 MONTHS

| | | | | | | | |
|---|---|---|---|---|---|---|---|
| COL4A3BP | P | 0.000 | 0.000 | 0.000 | 0.720 | 0.000 | 0.778 |
| MBNL2 | P | 0.000 | 0.000 | 0.000 | 0.676 | 0.000 | 0.757 |
| FABP4 | P | 0.001 | 0.001 | 0.023 | 0.195 | 0.001 | 0.703 |
| NEK1 | P | 0.001 | 0.002 | 0.004 | 0.673 | 0.006 | 0.699 |
| SKAP2 | P | 0.023 | 0.043 | 0.057 | 0.438 | 0.047 | 0.530 |
| COL4A1 | H | 0.000 | 0.003 | 0.003 | −0.297 | 0.007 | 0.691 |
| UBE2C | H | 0.000 | 0.000 | 0.000 | −0.409 | 0.000 | 0.767 |
| BIRC5 | H | 0.002 | 0.004 | 0.024 | −0.521 | 0.006 | 0.682 |
| COL18A1 | H | 0.095 | 0.092 | 0.046 | −0.252 | 0.119 | 0.607 |
| KPNA2 | H | 0.001 | 0.001 | 0.003 | −0.624 | 0.003 | 0.707 |
| MSN | H | 0.000 | 0.068 | 0.055 | −0.205 | 0.172 | 0.616 |
| ACTA2 | H | 0.115 | 0.137 | 0.116 | −0.179 | 0.178 | 0.595 |
| CDC25B | H | 0.011 | 0.004 | 0.001 | −0.524 | 0.007 | 0.685 |

36 MONTHS

| | | | | | | | |
|---|---|---|---|---|---|---|---|
| COL4A3BP | P | 0.000 | 0.000 | 0.000 | 0.716 | 0.000 | 0.785 |
| MBNL2 | P | 0.000 | 0.000 | 0.000 | 0.684 | 0.000 | 0.746 |
| FABP4 | P | 0.001 | 0.001 | 0.011 | 0.172 | 0.001 | 0.688 |
| NEK1 | P | 0.006 | 0.005 | 0.005 | 0.544 | 0.016 | 0.667 |
| SKAP2 | P | 0.030 | 0.052 | 0.084 | 0.408 | 0.044 | 0.693 |
| COL4A1 | H | 0.000 | 0.000 | 0.000 | −0.360 | 0.000 | 0.740 |
| UBE2C | H | 0.000 | 0.000 | 0.000 | −0.322 | 0.001 | 0.724 |
| BIRC5 | H | 0.010 | 0.006 | 0.023 | −0.443 | 0.012 | 0.663 |
| COL18A1 | H | 0.009 | 0.009 | 0.004 | −0.349 | 0.016 | 0.654 |
| KPNA2 | H | 0.007 | 0.005 | 0.019 | −0.552 | 0.006 | 0.666 |
| MSN | H | 0.005 | 0.003 | 0.004 | −0.305 | 0.021 | 0.678 |
| ACTA2 | H | 0.011 | 0.012 | 0.008 | −0.251 | 0.032 | 0.649 |
| CDC25B | H | 0.012 | 0.005 | 0.002 | −0.502 | 0.007 | 0.669 |

60 MONTHS

| | | | | | | | |
|---|---|---|---|---|---|---|---|
| COL4A3BP | P | 0.006 | 0.001 | 0.000 | 0.528 | 0.003 | 0.722 |
| MBNL2 | P | 0.006 | 0.001 | 0.002 | 0.430 | 0.003 | 0.719 |
| FABP4 | P | 0.002 | 0.001 | 0.002 | 0.142 | 0.001 | 0.719 |
| NEK1 | P | 0.337 | 0.311 | 0.429 | 0.381 | 0.156 | 0.567 |
| SKAP2 | P | 0.004 | 0.004 | 0.012 | 0.5911 | 0.005 | 0.590 |
| COL4A1 | H | 0.007 | 0.001 | 0.000 | −0.218 | 0.011 | 0.717 |
| UBE2C | H | 0.001 | 0.000 | 0.001 | −0.262 | 0.001 | 0.735 |
| BIRC5 | H | 0.016 | 0.007 | 0.011 | −0.447 | 0.009 | 0.679 |
| COL18A1 | H | 0.003 | 0.001 | 0.000 | −0.289 | 0.016 | 0.718 |
| KPNA2 | H | 0.014 | 0.009 | 0.002 | −0.515 | 0.004 | 0.673 |
| MSN | H | 0.071 | 0.014 | 0.011 | −0.180 | 0.129 | 0.663 |
| ACTA2 | H | 0.015 | 0.016 | 0.012 | −0.266 | 0.023 | 0.660 |
| CDC25B | H | 0.025 | 0.011 | 0.002 | −0.429 | 0.018 | 0.669 |

*P = protective marker; H = harmful marker

TABLE 5
Statistics associated with the Sig All 5.2 signature for determining the
likelihood of bladder cancer progression or non-progression.

| | | Wilcoxon signed- | | | | |
|---|---|---|---|---|---|---|
| Signature | T Test | rank test | KS Test | Cox Regression Analysis | | ROC |
| Sig All 5.2 | P-value | P-value | P-value | Beta coefficient | P-value | AUC |
| 24 Months | 1.57E−009 | 7.03E−007 | 3.09E−006 | 0.384 | 0.0000 | 0.815 |
| 36 Months | 2.55E−008 | 7.52E−008 | 3.40E−008 | 0.384 | 0.0000 | 0.818 |
| 60 Months | 1.20E−005 | 7.54E−006 | 2.93E−008 | 0.249 | 0.0001 | 0.797 |

### EXAMPLE 6

### Use of Assay to Predict Outcomes and Treatment Regimens

According to the invention, the expression levels of favorable markers will be decreased compared to their relative expression levels in a control and the expression levels of harmful markers will be increased compared to their relative expression in a control when the bladder cancer is likely to progress. When using QPCR or QRT-PCR, the expression levels of markers will be measured as Ct values. As Ct values correlate inversely with the base-2 logarithm of the mRNA concentration, decreased expression levels are seen as increased Ct values and increased expression levels are measured as lower Ct values. A higher Ct means less mRNA was present in the original sample. Thus, when using the signatures to predict a patient's outcome a greater difference between the Ct of the favorable markers minus the Ct of the unfavorable markers (a higher score), indicates a worse prognosis or a greater likelihood of progression; and, when this difference is lesser (a lower score), it indicates a better prognosis or greater likelihood of non-progression.

Strong positive correlation for progression and non-progression was found for signature Sig All 5.2. The signature shown in Table 3, Sig All 5.2, would allow clinicians to classify patients as high risk (HR) or low risk (LR) and modify treatment plans based upon this classification. It is recognized that in addition to the determination of progression as described herein, other factors may enter this analysis. Clinical practice may evolve over time, aiding in such analysis, and the methodology for classifying patients as HR or LR (as described above) may change. Moreover, there are other clinical measurements, like tumor grade, and size and location of tumor that clinicians use to predict risk of progression. The application of the signatures and scores of this invention, to classify patients as likely progressors or non-progressors, is a useful aid for clinicians in assessing risk of bladder cancer progression and can be combined with other clinical assessments to determine the best course of treatment.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for treating stage Ta or T1 bladder cancer, comprising:
a. determining the likelihood of progression of an individual's bladder cancer, by, determining in a bladder tumor sample from the individual:
i. the level of gene expression from at least one of the protective markers COL4A3BP, MBNL2, FABP4, NEK1 and SKAP2, wherein if the expression level determined for said protective markers is increased as compared to their expression level in a control or different bladder cancer sample, it indicates a decreased risk of progression relative to said control or different bladder cancer sample; and wherein if the expression level for said protective markers is decreased as compared to their expression level in a control or different bladder cancer sample, it indicates an increased risk of progression relative to said control or different bladder cancer sample; and
b. administering a therapeutic agent to the individual.

2. The method of claim 1 wherein BCG and/or a chemotherapeutic agent is administered to the individual if the expression level determined for said protective markers is decreased as compared to their expression level in a control or different bladder cancer sample.

3. The method of claim 1 wherein the method further includes determining, in the bladder tumor sample, the level of gene expression for the harmful markers COL4A1, UBE2C, BIRC5, COL18A1, KPNA2, MSN, and CDC25B wherein if the level determined for any harmful marker is increased as compared to their respective relative expression levels in a control or different bladder cancer sample, it indicates an increased risk of progression relative to said control or different bladder cancer sample; and wherein if the expression level for any harmful marker is decreased as compared to their respective relative expression levels in a control or different bladder cancer sample, it indicates a decreased risk of progression relative to said control or different bladder cancer sample.

4. A method for treating stage Ta or T1 bladder cancer, comprising:
a. determining the likelihood of progression of an individual's bladder cancer, by, determining in a bladder tumor sample from the individual:
i. the level of gene expression for at least one of the harmful markers COL4A1, UBE2C, BIRC5, COL18A1, KPNA2, MSN, and CDC25B, wherein if the expression levels for any harmful marker is increased compared to their relative expression levels in a control or different bladder cancer sample, this indicates an increased risk of progression; and
b. administering a therapeutic agent to the individual.

5. The method of claim 4 wherein if the harmful marker expression levels indicate an increased risk of progression, BCG or a chemotherapeutic agent is administered to the individual.

6. A method for treating stage Ta or T1 bladder cancer, comprising:
a. determining the likelihood of progression of an individual's bladder cancer, by, determining in a bladder tumor sample from the individual:
i. the level of gene expression for the markers COL4A1, UBE2C, COL4A3BP, MBNL2, and FABP4, wherein if the expression levels for either COL4A1 or both COL4A1 and UBE2C are higher than the expression levels for COL4A3BP, MBNL2 and/or FABP4 as compared to their respective relative expression levels in a control or different bladder cancer sample, this indicates an increased risk of progression; and
b. administering a therapeutic agent to the individual.

7. The method of claim 6 wherein if the marker expression levels indicate an increased risk of progression, BCG is administered to the individual.

8. The method of claim 4 wherein the expression levels for one or more of the protective markers COL4A3BP, MBNL2, FABP4, NEK1 and SKAP2, are determined, whereby if the expression levels for the harmful markers are increased, as compared to their respective relative expression levels in a control or different bladder cancer sample, and if the expression levels for the protective markers are decreased, as compared to their respective relative expression levels in a control or different bladder cancer sample, it indicates an increased risk of progression.

9. A method of claim 6 wherein the expression levels for one or more of the markers BIRC5, COL18A1, CDC25B, SKAP2, MSN, NEK1, and KPNA2 are determined, whereby if the expression levels for BIRC5, COL18A1, COL4A1, MSN, KPNA2, CDC25B, and/or UBE2C are decreased relative to the expression levels for COL4A3BP, MBNL2, FABP4, NEK1, and/or SKAP2, as compared to their respective relative expression levels in a control or different bladder cancer sample, it indicates a decreased risk of progression relative to said control or different bladder cancer sample.

10. The method of claim 8 wherein if the harmful and protective marker expression levels indicate an increased risk of progression, BCG or a chemotherapeutic agent is administered to the individual.


SEQUENCE LISTING (120 sequences)

SEQ ID NO: 1 - DNA, 26 bp, Artificial Sequence
  Description of Artificial Sequence Synthetic ACTA2 forward primer
gtctctagcacacaactgtgaatgtc

SEQ ID NO: 2 - DNA, 25 bp, Artificial Sequence
  Description of Artificial Sequence Synthetic ACTA2 reverse primer
ctaggaatgatttggaaaagaactg

SEQ ID NO: 3 - DNA, 68 bp, Artificial Sequence
  Description of Artificial Sequence Synthetic ACTA2 oligonucleotide
gtctctagcacacaactgtgaatgtcctgtggaattatgccttcagttcttttccaaatcattcctag

SEQ ID NO: 4 - DNA, 26 bp, Artificial Sequence
  Description of Artificial Sequence Synthetic ADAM10 forward primer
cagtattacttatgggaattgctctg

SEQ ID NO: 5 - DNA, 27 bp, Artificial Sequence
  Description of Artificial Sequence Synthetic ADAM10 reverse primer
ttggattactacttggagtatgaacac

SEQ ID NO: 6 - DNA, 87 bp, Artificial Sequence
  Description of Artificial Sequence Synthetic ADAM10 oligonucleotide
cagtattacttatgggaattgctctgatcatgctaatggctggatttattaagatatgcagtgttcatactccaagtagtaatccaa

SEQ ID NO: 7 - DNA, 20 bp, Artificial Sequence
  Description of Artificial Sequence Synthetic AURKB forward primer
tttgctatgagctgctggtg

SEQ ID NO: 8 - DNA, 23 bp, Artificial Sequence
  Description of Artificial Sequence Synthetic AURKB reverse primer
actttaggtccaccttgacgatg

SEQ ID NO: 9 - DNA, 90 bp, Artificial Sequence
  Description of Artificial Sequence Synthetic AURKB oligonucleotide
tttgctatgagctgctggtggggaacccaccctttgagagtgcatcacacaacgagacctatcgccgcatcgtcaaggtggacctaaagt

SEQ ID NO: 10 - DNA, 23 bp, Artificial Sequence
  Description of Artificial Sequence Synthetic BIRC5 forward primer
ctgaagtctggcgtaagatgatg

SEQ ID NO: 11 - DNA, 22 bp, Artificial Sequence
  Description of Artificial Sequence Synthetic BIRC5 reverse primer
gaagctgtaacaatccaccctg

SEQ ID NO: 12 - DNA, 79 bp, Artificial Sequence
  Description of Artificial Sequence Synthetic BIRC5 oligonucleotide
ctgaagtctggcgtaagatgatggatttgattcgccctcctccctgtcatagagctgcagggtggattgttacagcttc

SEQ ID NO: 13 - DNA, 26 bp, Artificial Sequence
  Description of Artificial Sequence Synthetic C10ORF58 forward primer
gtaaacctactttctgttctggaagc

SEQ ID NO: 14 - DNA, 22 bp, Artificial Sequence
  Description of Artificial Sequence Synthetic C10ORF58 reverse primer
ttttctctgaggccaaagtctg

SEQ ID NO: 15 - DNA, 67 bp, Artificial Sequence
  Description of Artificial Sequence Synthetic C10ORF58 oligonucleotide
gtaaacctactttctgttctggaagctgctaagatgatcaaaccacagactttggcctcagagaaaa

SEQ ID NO: 16 - DNA, 21 bp, Artificial Sequence
  Description of Artificial Sequence Synthetic CDC20 forward primer
agtccaatgtcctggcaacag

SEQ ID NO: 17 - DNA, 21 bp, Artificial Sequence
  Description of Artificial Sequence Synthetic CDC20 reverse primer
ccagagcacacattccagatg

SEQ ID NO: 18 - DNA, 70 bp, Artificial Sequence
  Description of Artificial Sequence Synthetic CDC20 oligonucleotide
agtccaatgtcctggcaacaggagggggcaccagtgatcgacacattcgcatctggaatgtgtgctctgg

SEQ ID NO: 19 - DNA, 20 bp, Artificial Sequence
  Description of Artificial Sequence Synthetic CDC25B forward primer
gatggaaggttggatggatg

SEQ ID NO: 20 - DNA, 20 bp, Artificial Sequence
  Description of Artificial Sequence Synthetic CDC25B reverse primer
acctggtttgggtatgcaag

SEQ ID NO: 21 - DNA, 76 bp, Artificial Sequence
  Description of Artificial Sequence Synthetic CDC25B oligonucleotide
gatggaaggttggatggatgggtggatggccgtggatggccgtggatgcgcagtgccttgcatacccaaaccaggt

SEQ ID NO: 22 - DNA, 27 bp, Artificial Sequence
  Description of Artificial Sequence Synthetic CDH5 forward primer
aaacaattcctgtaaccttctattttc

SEQ ID NO: 23 - DNA, 20 bp, Artificial Sequence
  Description of Artificial Sequence Synthetic CDH5 reverse primer
cttgtcatgcaccagtttgg

SEQ ID NO: 24 - DNA, 90 bp, Artificial Sequence
  Description of Artificial Sequence Synthetic CDH5 oligonucleotide
aaacaattcctgtaaccttctattttctataattgtagtaattgctctacagataatgtctatatattggccaaactggtgcatgacaag

SEQ ID NO: 25 - DNA, 26 bp, Artificial Sequence
  Description of Artificial Sequence Synthetic CDKN3 forward primer
atctctaccagcaatgtggaattatc

SEQ ID NO: 26 - DNA, 22 bp, Artificial Sequence
  Description of Artificial Sequence Synthetic CDKN3 reverse primer
ctatgtcaggagtccctccatc

SEQ ID NO: 27 - DNA, 69 bp, Artificial Sequence
  Description of Artificial Sequence Synthetic CDKN3 oligonucleotide
atctctaccagcaatgtggaattatcacccatcatcatccaatcgcagatggagggactcctgacatag

SEQ ID NO: 28 - DNA, 22 bp, Artificial Sequence
  Description of Artificial Sequence Synthetic COL18A1 forward primer
gggctggttctgtaattgtgtg

SEQ ID NO: 29 - DNA, 25 bp, Artificial Sequence
  Description of Artificial Sequence Synthetic COL18A1 reverse primer
aaaaggtcacttttatttgcctgtc

SEQ ID NO: 30 - DNA, 65 bp, Artificial Sequence
  Description of Artificial Sequence Synthetic COL18A1 oligonucleotide
gggctggttctgtaattgtgtgtgatgtgaagccaattcagacaggcaaataaaagtgacctttt

SEQ ID NO: 31 - DNA, 23 bp, Artificial Sequence
  Description of Artificial Sequence Synthetic COL4A1 forward primer
ctgcctggaggagtttagaagtg

SEQ ID NO: 32 - DNA, 24 bp, Artificial Sequence
  Description of Artificial Sequence Synthetic COL4A1 reverse primer
ctgtaagcgtttgcgtagtaattg

SEQ ID NO: 33 - DNA, 81 bp, Artificial Sequence
  Description of Artificial Sequence Synthetic COL4A1 oligonucleotide
ctgcctggaggagtttagaagtgcgccattcatcgagtgtcacggccgtgggacctgcaattactacgcaaacgcttacag

SEQ ID NO: 34 - DNA, 22 bp, Artificial Sequence
  Description of Artificial Sequence Synthetic COL4A3BP forward primer
tttctgtggatcatgacagtgc

SEQ ID NO: 35 - DNA, 24 bp, Artificial Sequence
  Description of Artificial Sequence Synthetic COL4A3BP reverse primer
caaggtttgacaaatcatagcaac

SEQ ID NO: 36 - DNA, 83 bp, Artificial Sequence
  Description of Artificial Sequence Synthetic COL4A3BP oligonucleotide
tttgtaatttttctgtggatcatgacagtgctcctctaaacaaccgatgtgtccgtgccaaaataaatgttgctatgatttgt

SEQ ID NO: 37 - DNA, 26 bp, Artificial Sequence
  Description of Artificial Sequence Synthetic CPS1 forward primer
ggaagtaaggttcattcccttaagac

SEQ ID NO: 38 - DNA, 26 bp, Artificial Sequence
  Description of Artificial Sequence Synthetic CPS1 reverse primer
ccttacagtgggtggaattaatagtg

SEQ ID NO: 39 - DNA, 86 bp, Artificial Sequence
  Description of Artificial Sequence Synthetic CPS1 oligonucleotide
ggaagtaaggttcattcccttaagacgatggattctgttgaactatggggtcccacactgcactattaattccacccactgtaagg

SEQ ID NO: 40 - DNA, 22 bp, Artificial Sequence
  Description of Artificial Sequence Synthetic DCTD forward primer
tgtgccccttctctttaatctc

SEQ ID NO: 41 - DNA, 22 bp, Artificial Sequence
  Description of Artificial Sequence Synthetic DCTD reverse primer
gaaagccttttctcaacacagg

SEQ ID NO: 42 - DNA, 72 bp, Artificial Sequence
  Description of Artificial Sequence Synthetic DCTD oligonucleotide
tgtgccccttctctttaatctcatttaatttttattaaacatgctcagtacctgtgttgagaaaaggctttc

SEQ ID NO: 43 - DNA, 26 bp, Artificial Sequence
  Description of Artificial Sequence Synthetic FABP4 forward primer
agagaaaacgagaggatgataaactg

SEQ ID NO: 44 - DNA, 26 bp, Artificial Sequence
  Description of Artificial Sequence Synthetic FABP4 reverse primer
cttatgctctctcataaactctcgtg

SEQ ID NO: 45 - DNA, 84 bp, Artificial Sequence
  Description of Artificial Sequence Synthetic FABP4 oligonucleotide
agagaaaacgagaggatgataaactggtggtggaatgcgtcatgaaaggcgtcacttccacgagagtttatgagagagcataag

SEQ ID NO: 46 - DNA, 25 bp, Artificial Sequence
  Description of Artificial Sequence Synthetic GAPDH forward primer
ttacatgttccaatatgattccacc

SEQ ID NO: 47 - DNA, 23 bp, Artificial Sequence
  Description of Artificial Sequence Synthetic GAPDH reverse primer
atttccattgatgacaagcttcc

SEQ ID NO: 48 - DNA, 85 bp, Artificial Sequence
  Description of Artificial Sequence Synthetic GAPDH oligonucleotide
ttacatgttccaatatgattccacccatggcaaattccatggcaccgtcaaggctgagaacgggaagcttgtcatcaatggaaat

SEQ ID NO: 49 - DNA, 20 bp, Artificial Sequence
  Description of Artificial Sequence Synthetic IER2 forward primer
cttgccagggagtttctgag

SEQ ID NO: 50 - DNA, 25 bp, Artificial Sequence
  Description of Artificial Sequence Synthetic IER2 reverse primer
atttctaacaaaacgccaggtagac

SEQ ID NO: 51 - DNA, 90 bp, Artificial Sequence
  Description of Artificial Sequence Synthetic IER2 oligonucleotide
cttgccagggagtttctgagggtctgctttgtttacctttcgtgcggtggattctttttaactccgtctacctggcgttttgttagaaat

SEQ ID NO: 52 - DNA, 22 bp, Artificial Sequence
  Description of Artificial Sequence Synthetic IGF2 forward primer
catcgttgaggagtgctgtttc

SEQ ID NO: 53 - DNA, 22 bp, Artificial Sequence
  Description of Artificial Sequence Synthetic IGF2 reverse primer
gggtagcacagtacgtctccag

SEQ ID NO: 54 - DNA, 65 bp, Artificial Sequence
  Description of Artificial Sequence Synthetic IGF2 oligonucleotide
catcgttgaggagtgctgtttccgcagctgtgacctggccctcctggagacgtactgtgctaccc

SEQ ID NO: 55 - DNA, 22 bp, Artificial Sequence
  Description of Artificial Sequence Synthetic ITGB4 forward primer
catcatccctgacatccctatc

SEQ ID NO: 56 - DNA, 25 bp, Artificial Sequence
  Description of Artificial Sequence Synthetic ITGB4 reverse primer
gtagaacgtcatcgctgtacataag

SEQ ID NO: 57 - DNA, 83 bp, Artificial Sequence
  Description of Artificial Sequence Synthetic ITGB4 oligonucleotide
catcatccctgacatccctatcgtggacgcccagagcggggaggactacgacagcttccttatgtacagcgatgacgttctac

SEQ ID NO: 58 - DNA, 25 bp, Artificial Sequence
  Description of Artificial Sequence Synthetic KPNA2 forward primer
gcagattttaagacacaaaaggaag

SEQ ID NO: 59 - DNA, 26 bp, Artificial Sequence
  Description of Artificial Sequence Synthetic KPNA2 reverse primer
aaggtacacaatctgttcaactgttc

SEQ ID NO: 60 - DNA, 84 bp, Artificial Sequence
  Description of Artificial Sequence Synthetic KPNA2 oligonucleotide
gcagattttaagacacaaaaggaagctgtgtgggccgtgaccaactataccagtggtggaacagttgaacagattgtgtacctt

SEQ ID NO: 61 - DNA, 22 bp, Artificial Sequence
  Description of Artificial Sequence Synthetic LBR forward primer
atcagaaagtggtggcgttttc

SEQ ID NO: 62 - DNA, 25 bp, Artificial Sequence
  Description of Artificial Sequence Synthetic LBR reverse primer
ttaccagggaaagaatttaatgtcc

SEQ ID NO: 63 - DNA, 88 bp, Artificial Sequence
  Description of Artificial Sequence Synthetic LBR oligonucleotide
atcagaaagtggtggcgttttctgtactggattgcaccaaggaagcttttggggaggaaggaaggacattaaattctttccctggtaa

SEQ ID NO: 64 - DNA, 23 bp, Artificial Sequence
  Description of Artificial Sequence Synthetic LGALS1 forward primer
ctgaatctcaaacctggagagtg

SEQ ID NO: 65 - DNA, 22 bp, Artificial Sequence
  Description of Artificial Sequence Synthetic LGALS1 reverse primer
ggttcagcacgaagctcttagc

SEQ ID NO: 66 - DNA, 76 bp, Artificial Sequence
  Description of Artificial Sequence Synthetic LGALS1 oligonucleotide
ctgaatctcaaacctggagagtgccttcgagtgcgaggcgaggtggctcctgacgctaagagcttcgtgctgaacc

SEQ ID NO: 67 - DNA, 26 bp, Artificial Sequence
  Description of Artificial Sequence Synthetic MAT2B forward primer
ttgtctaaagaaactaaagggcagtc

SEQ ID NO: 68 - DNA, 24 bp, Artificial Sequence
  Description of Artificial Sequence Synthetic MAT2B reverse primer
agtttagccaggacaaacaaaatg

SEQ ID NO: 69 - DNA, 81 bp, Artificial Sequence
  Description of Artificial Sequence Synthetic MAT2B oligonucleotide
ttgtctaaagaaactaaagggcagtcatgccctgtttgcagtaatttttctttttatcattttgtttgtcctggctaaact

SEQ ID NO: 70 - DNA, 22 bp, Artificial Sequence
  Description of Artificial Sequence Synthetic MBNL2 forward primer
acttcatccagtgcccactttc

SEQ ID NO: 71 - DNA, 23 bp, Artificial Sequence
  Description of Artificial Sequence Synthetic MBNL2 reverse primer
ggggttacaggtgctaggtaagg

SEQ ID NO: 72 - DNA, 90 bp, Artificial Sequence
  Description of Artificial Sequence Synthetic MBNL2 oligonucleotide
acttcatccagtgcccactttccctgtaggtcccgcgatagggacaaatacggctattagctttgctccttacctagcacctgtaacccc

SEQ ID NO: 73 - DNA, 26 bp, Artificial Sequence
  Description of Artificial Sequence Synthetic MCM7 forward primer
gagatgtcaaaggactctcttctagg

SEQ ID NO: 74 - DNA, 22 bp, Artificial Sequence
  Description of Artificial Sequence Synthetic MCM7 reverse primer
gcaaatatcacatctgctggtc

SEQ ID NO: 75 - DNA, 77 bp, Artificial Sequence
  Description of Artificial Sequence Synthetic MCM7 oligonucleotide
gagatgtcaaaggactctcttctaggagacaaggggcagacagctaggactcagagaccagcagatgtgatatttgc

SEQ ID NO: 76 - DNA, 22 bp, Artificial Sequence
  Description of Artificial Sequence Synthetic MSN forward primer
cctgaccttgaggagtcttgtg

SEQ ID NO: 77 - DNA, 26 bp, Artificial Sequence
  Description of Artificial Sequence Synthetic MSN reverse primer
aatataggacatatcaccaagtgagc

SEQ ID NO: 78 - DNA, 65 bp, Artificial Sequence
  Description of Artificial Sequence Synthetic MSN oligonucleotide
cctgaccttgaggagtcttgtgtgcattgctgtgaattagctcacttggtgatatgtcctatatt

SEQ ID NO: 79 - DNA, 26 bp, Artificial Sequence
  Description of Artificial Sequence Synthetic NEK1 forward primer
ctaaaagaccagcttcaggacaaaac

SEQ ID NO: 80 - DNA, 25 bp, Artificial Sequence
  Description of Artificial Sequence Synthetic NEK1 reverse primer
ctaaaggtattccatatttagcggc

SEQ ID NO: 81 - DNA, 90 bp, Artificial Sequence
  Description of Artificial Sequence Synthetic NEK1 oligonucleotide
ctaaaagaccagcttcaggacaaaactcgatttctgttatgcctgctcagaaaattacaaagcctgccgctaaatatggaatacctttag

SEQ ID NO: 82 - DNA, 22 bp, Artificial Sequence
  Description of Artificial Sequence Synthetic NR1H3 forward primer
ggaattcatcaaccccatcttc

SEQ ID NO: 83 - DNA, 22 bp, Artificial Sequence
  Description of Artificial Sequence Synthetic NR1H3 reverse primer
gatagcaatgagcaaggcaaac

SEQ ID NO: 84 - DNA, 88 bp, Artificial Sequence
  Description of Artificial Sequence Synthetic NR1H3 oligonucleotide
ggaattcatcaaccccatcttcgagttctccagggccatgaatgagctgcaactcaatgatgccgagtttgccttgctcattgctatc

SEQ ID NO: 85 - DNA, 25 bp, Artificial Sequence
  Description of Artificial Sequence Synthetic PEA15 forward primer
actccttatattgctgtgagattgc

SEQ ID NO: 86 - DNA, 24 bp, Artificial Sequence
  Description of Artificial Sequence Synthetic PEA15 reverse primer
acctttattccgggttagaacaag

SEQ ID NO: 87 - DNA, 86 bp, Artificial Sequence
  Description of Artificial Sequence Synthetic PEA15 oligonucleotide
actccttatattgctgtgagattgcccctatcttgtgctcttctgtctgcagtgtgcacggccttgttctaacccggaataaaggt

SEQ ID NO: 88 - DNA, 27 bp, Artificial Sequence
  Description of Artificial Sequence Synthetic PPP2R5C forward primer
gtactacattgaaaataaaccggtgac

SEQ ID NO: 89 - DNA, 25 bp, Artificial Sequence
  Description of Artificial Sequence Synthetic PPP2R5C reverse primer
tacattttggaaagagtgaagatgc

SEQ ID NO: 90 - DNA, 80 bp, Artificial Sequence
  Description of Artificial Sequence Synthetic PPP2R5C oligonucleotide
gtactacattgaaaataaaccggtgactgtttttcttcataaagttctgcgtttggcatcttcactctttccaaaatgta

SEQ ID NO: 91 - DNA, 26 bp, Artificial Sequence
  Description of Artificial Sequence Synthetic SKAP2 forward primer
tggagatgtatgatatttgagagtcc

SEQ ID NO: 92 - DNA, 23 bp, Artificial Sequence
  Description of Artificial Sequence Synthetic SKAP2 reverse primer
ctaaatccaaagcatttgcagac

SEQ ID NO: 93 - DNA, 3984 bp, Homo sapiens
  src kinase associated phosphoprotein 2 (SKAP2), mRNA
ggaaaaccgaggaatacacatgcgcagttggacccctcaggcccttcgtgtcccttcccacccatctccccgccccggccctctgggcggggctgggccgacagtccagctgcagctcgctggagattcagtgacttccttgttgtgagccccggcccggcagtgtcccgactcgtagccccgctgttcttaatccgggccgctagcctgagtctaggtcgcagccgcagccccaccccgtcggtcaccttttcagcgcaggtcctttccccgcacgccctgcgctccctaacatgcccaaccccagcagcacctcctctccctaccccctccctgaggaaattaggaacctgttggcagatgttgaaacatttgtagcagatatactgaaaggagaaaatttatccaagaaagcaaaggaaaagagagaatcccttattaagaagataaaagatgtaaagtctatctatcttcaggaatttcaagacaaaggtgatgcagaagatggggaagaatatgatgacccttttgctgggcctccagacactatttcattagcctcagaacgatatgataaagacgatgaagccccctctgatggagcccagtttcctccaattgcagcacaagaccttccttttgttctaaaggctggctaccttgaaaaacgcagaaaagatcacagctttctgggatttgaatggcagaaacggtggtgtgctctcagtaaaacggtattctattattatggaagtgataaagacaaacaacagaaaggtgaatttgcaatagatggctacagtgtcagaatgaataacactctaagaaaggatggaaagaaagattgctgttttgaaatctctgctcctgataaacgtatatatcagtttacagcagcttctcccaaagatgctgaagaatgggtacagcagctgaaatttgtattgcaagatatggaatctgatattattcctgaggattatgatgagagaggagaattatatgatgatgttgatcatcctctaccaataagcaatccactaacaagcagtcaaccaatagatgatgaaatttatgaagaacttccagaagaagaagaggacagtgctccagtgaaagtggaagaacaaaggaagatgagtcaggatagtgtccatcacacctcaggggataagagcactgattatgctaatttttaccagggattgtgggattgtactggagctttttctgatgagttgtcatttaagcgtggtgatgtgatttacattcttagcaaggaatacaatagatatggctggtgggtaggagaaatgaagggagccattggcttggtgcctaaagcctacataatggagatgtatgatatttgagagtcctggaaaaggaaaattcttctgcttgtctgcaaatgctttggatttagaagcgtcatgaaagcacgagtgacagctcctaacctctccttgttttattaaacattacttatctttgactgttattttatgcagtcgctcattaaaatattcctctgatgtgaaattaaatgaaggatattaatgtaaattagatgcaaccagttaagttatacctgttgctattttgcaaagaaataattatagtttttatttacccatttgatttgtgtgaagaattcatcactattttatacgtaacatatagtctactatagcatagtatgctactattgctacttctggtgtgatttgtaatgtttcttaatcattggacatcaattatttttagagagtaatgtataatttcatagcattttaaatttagtgtatcatgcgagttttttttggtagatgctgaagaatgtggttgctaaacaaagaatgctaaagaatgttcaaactttatagataactttattgttattatttattttcacacatttaattcctattaagtacagccgcaagaaagaaaaaatgatgaagttgcaaatggcagtgtgctgtcacctgcaacagaagtgcgtaccagaagtatgactcatgcaaagcattttaccgtacaaatatcctggtgcgatgatgggcctggcaacattttctactgtacttttgtttaaatttaatgaaacaaaaaattcctaagaaataccaccctaccactaacaaaatggtataaagaatctcccagccaggccaacatggtgaaaccctgtctctactaaaaattagccaagtgtggtaatgttcacctgcagtcccagctacttgggaggccaaggcacgagaattgcttgaacaaggtaggcagaggttgcagtgagccaagatcgcgccactgcactccagcctgggcgacagagtgagactccatctcagaaaaaaaaaaaaaaaaaaacagaatctcagtgtattctcaaagtaaaaaggcataaccaagcactctcttatcttgccttattgctatactatttacatcccactgcagaacagcagatttgaggcttttctatatacttctcagagcactgaaaagaaaggaagggtttgcagaggagagtgtagaaatcccagtggtagcatgtaccaacaggtgagtagaaaggtagtgttagcctgacatttgagttatactctgtgctgcctgagcaagatttgtagaatcatataattaccttttcatgtatatttgaaatcagaggtgtttaaaatacctatgagataccaatgtagccttaacatatgtcaaaatgcattgctggttagataattatttggactacacataaactcctaatattgaatcattacctatcaggtattatctttatggaacttttcaatatctttgctttataaagattctaaacatgtatctgagctggtaatattttaaaatctccattatttgtgtaaaactgtttataagcagtgtttgagagggtctgctttaccattaccccctcaatatcatgatcatccaatctcaaatgtgaaaaaaaaaaagaaatttgattttaggtattgtgagtaaacaagtttatatagagagacattgtgaagttaaagttttcagaagttacatttgtgcagttcttaccttttcctcatatagtgccattgaaatagactgaaattatcttggcaaaagttagacaaccaaagacgactttagtggactggttttcaaaacttgagcagctgaaaagcaaaagccgttgtttcccatgacaatgtagcctttgtggatttgggtttgtgctttgggttgaaaagaagtttttagtcctaggccagtagatggcagcagcttttcattgcagacaaaacctcttgaaacccttcccccatggcacaaactcgcccatgatggaaagcatctagatttctgcctccttttacagttaatccaggagagggagtcctttgccaactgatgaccaacagttccaagccagatagtctcgtgaacagtgacaatacagaaataaggtgttatttctgttcagatctccaccggcctttgttcttttaaaacttgaatataggtgggagacataagaaaggaaagaaaagacttaaaactggagtgacaggacaaataatcattactttcaattcatgactgctttatattcatttgatgaaatcatttgtatacaaaccagggagagttttctttacacccttgacaatatatcacatactcttcaagatcataataatatcattaatataaatttaaacaacatggcttgttagaaaatatgctaattgctatggtctcattatgtttgcttagcttttatttgtttttctgtgaacagttagagagctaatttttttcaaaggtgattgtaagtcatattttatatagcattttgcttgattatttgctctgtactgaatttgtactctattgccattagatcttacaataatgttccactctgcaaatttttaaggttcaaataaagtttaattgtttgcaaaaaaaaaaaaaaaa

SEQ ID NO: 94 - DNA, 22 bp, Artificial Sequence
  Description of Artificial Sequence Synthetic SDC1 forward primer
agacaccttggacatcctcctc

SEQ ID NO: 95 - DNA, 22 bp, Artificial Sequence
  Description of Artificial Sequence Synthetic SDC1 reverse primer
taagcaagtaagtgcaggagcc

SEQ ID NO: 96 - DNA, 80 bp, Artificial Sequence
  Description of Artificial Sequence Synthetic SDC1 oligonucleotide
agacaccttggacatcctcctcccacccggctgcagaggccagaggcccccagcccagggctcctgcacttacttgctta

SEQ ID NO: 97 - DNA, 26 bp, Artificial Sequence
  Description of Artificial Sequence Synthetic SEC14L1 forward primer
tgtttctacctttagtaccttgccac

SEQ ID NO: 98 - DNA, 26 bp, Artificial Sequence
  Description of Artificial Sequence Synthetic SEC14L1 reverse primer
agtactaagaaatgggaaatgacagc

SEQ ID NO: 99 - DNA, 66 bp, Artificial Sequence
  Description of Artificial Sequence Synthetic SEC14L1 oligonucleotide
tgtttctacctttagtaccttgccactcttttaaaacgctgctgtcatttcccatttcttagtact

SEQ ID NO: 100 - DNA, 25 bp, Artificial Sequence
  Description of Artificial Sequence Synthetic TCF4 forward primer
gaatcacatgggacagatgtaaaag

SEQ ID NO: 101 - DNA, 26 bp, Artificial Sequence
  Description of Artificial Sequence Synthetic TCF4 reverse primer
aatacagctgttaaggaagtggtctc

SEQ ID NO: 102 - DNA, 84 bp, Artificial Sequence
  Description of Artificial Sequence Synthetic TCF4 oligonucleotide
gaatcacatgggacagatgtaaaagggtccaagttgccacattgcttcattaaaacaagagaccacttccttaacagctgtatt

SEQ ID NO: 103 - DNA, 26 bp, Artificial Sequence
  Description of Artificial Sequence Synthetic UBE2C forward primer
tctaggagaacccaacattgatagtc

SEQ ID NO: 104 - DNA, 25 bp, Artificial Sequence
  Description of Artificial Sequence Synthetic UBE2C reverse primer
tcttgcaggtacttcttaaaagctg

SEQ ID NO: 105 - DNA, 90 bp, Artificial Sequence
  Description of Artificial Sequence Synthetic UBE2C oligonucleotide
tctaggagaacccaacattgatagtcccttgaacacacatgctgccgagctctggaaaaaccccacagcttttaagaagtacctgcaaga

SEQ ID NO: 106 - DNA, 22 bp, Artificial Sequence
  Description of Artificial Sequence Synthetic WNT2B forward primer
ataagaaactgtgcaagctccc

SEQ ID NO: 107 - DNA, 24 bp, Artificial Sequence
  Description of Artificial Sequence Synthetic WNT2B reverse primer
tctactctcccttcaaatctccag

SEQ ID NO: 108 - DNA, 58 bp, Artificial Sequence
  Description of Artificial Sequence Synthetic WNT2B oligonucleotide
ataagaaactgtgcaagctccctgatttcccgctctggagatttgaagggagagtaga

SEQ ID NO: 109 - DNA, 6447 bp, Homo sapiens
  Homo sapiens collagen, type IV, alpha 1 (COL4A1), mRNA
aggtctccgcttggagccgccgcacccgggacggtgcgtatcgctggaagtccggccttccgagagctagctgtccgccgcggcccccgcacgccgggcagccgtccctcgcgcctcgggcgcgccaccatggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgcaggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccccgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgccagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccaggactgcgtggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggacaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataagaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagcttcaatagtggcataccaaatggcacttttgatgaaataaaatatcaatattttctgcaatccaatgcactgatgtgtgaagtgagaactccatcagaaaaccaaagggtgctaggaggtgtgggtgccttccatactgtttgcccattttcattcttgtattataattaattttctacccccagagataaatgtttgtttatatcactgtctagctgtttcaaaatttaggtcccttggtctgtacaaataatagcaatgtaaaaatggttttttgaacctccaaatggaattacagactcagtagccatatcttccaaccccccagtataaatttctgtctttctgctatgtgtggtactttgcagctgcttttgcagaaatcacaattttcctgtggaataaagatggtccaaaaatagtcaaaaattaaatatatatatatattagtaatttatatagatgtcagcaattaggcagatcaaggtttagtttaacttccactgttaaaataaagcttacatagttttcttcctttgaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagctt

SEQ ID NO: 110 - DNA, 5689 bp, Homo sapiens
  Homo sapiens NIMA (never in mitosis gene a)- related kinase 1 (NEK1), transcript variant 2, mRNA
cacacgccagcgccggtgacgcgccggccgctctcccttagtccgcattcgctccagggttttgggaccctaggttgcggagtccttaccctaccctggcctctcgagcagttgtccccataactcggaatctagagccgctgttgcgaggcaggagcacgtggcagtcaagtagcttcccagtcccgaacgccgcccgtccccaccccgccgtggccactagcaacgacctctgtgaagttggagaggcggtaacggaggcactccccctgctgcaccccgccgtttctacggggctcagaaaccagtttgtttgtttcgtcggggtagtgtcgacctgtcttacgggcgtcgcccgagacaggacggagtcaaacccgtggtatcaactgaagacgagtgtcaggtgtggagagtctcagtgccccctttcagtctggactgtgagctgctgctggttagacagtcttggtttctctttcaggatgtcattttcaaaatgcgggatggtacctctgctttattaagccccgtaggaagactgccacacctagactgatgcttattagtcatcaccgttattcctactaacgtcctgtgtcactgagttttttaaatgtctagcatatctgtaaagatgccttagaaaaagaatcatggagaagtatgttagactacagaagattggagaaggttcatttggaaaagccattcttgttaaatctacagaagatggcagacagtatgttatcaaggaaattaacatctcaagaatgtccagtaaagaaagagaagaatcaaggagagaagttgcagtattggcaaacatgaagcatccaaatattgtccagtatagagaatcatttgaagaaaatggctctctctacatagtaatggattactgtgagggaggggatctgtttaagcgaataaatgctcagaaaggcgttttgtttcaagaggatcagattttggactggtttgtacagatatgtttggccctgaaacatgtacatgatagaaaaattcttcatcgagacattaaatctcagaacatatttttaactaaagatggaacagtacaacttggagattttggaattgctagagttcttaatagtactgtagagctggctcgaacttgcatagggaccccatactacttgtcacctgaaatctgtgaaaacaaaccttacaataataaaagtgacatttgggctctggggtgtgtcctttatgagctgtgtacacttaaacatgcttttgaagctggcagtatgaaaaacctggtactgaagataatatctggatcttttccacctgtgtctttgcattattcctatgatctccgcagtttggtgtctcagttatttaaaagaaatcctagggatagaccatcagtcaactccatattggagaaaggttttatagccaaacgcattgaaaagtttctctctcctcagcttattgcagaagaattttgtctaaaaacattttcgaagtttggatcacagcctataccagctaaaagaccagcttcaggacaaaactcgatttctgttatgcctgctcagaaaattacaaagcctgccgctaaatatggaatacctttagcatataagaaatatggagataaaaaattacacgaaaagaaaccactgcaaaaacataaacaggcccatcaaactccagagaagagagtgaatactggagaagaaaggaggaaaatatctgaggaagcagcaagaaagagaaggctggaatttattgaaaaagaaaagaaacaaaaggatcagattattagtttaatgaaggctgaacaaatgaaaaggcaagaaaaggaaaggttggaaagaataaatagggccagggaacaaggatggagaaatgtgctaagtgctggtggaagtggtgaagtaaaggctccttttctgggcagtggagggactatagctccatcatctttttcttctcgaggacagtatgaacattaccatgccatttttgaccaaatgcagcaacaaagagcagaagataatgaagctaaatggaaaagagaaatatatggtcgaggtcttccagaaagaggaattctgcctggagttcgtccaggatttccttatggggctgcaggtcatcaccattttcctgatgctgatgatattagaaaaactttgaaaagattgaaggcggtgtctaaacaagccaatgcaaacaggcaaaaagggcagctagctgtagaaagagctaaacaagtagaagagttcctgcagcgaaaacgggaagctatgcagaataaagctcgagccgaaggacatatggtttatctggcaagactgaggcaaataagactacagaatttcaatgagcgccaacagattaaagccaaacttcgtggtgaaaagaaagaagctaatcattctgaaggacaagaaggaagtgaagaggctgacatgaggcgcaaaaaaatcgaatcactgaaggcccatgcaaatgcacgtgctgctgtactaaaagaacaactagaacgaaagagaaaggaggcttatgagagagaaaaaaaagtgtgggaagagcatttggtggctaaaggagttaagagttctgatgtttctccacctttgggacagcatgaaacaggtggctctccatcaaagcaacagatgagatctgttatttctgtaacttcagctttgaaagaagttggcgtggacagtagtttaactgatacccgggaaacttcagaagagatgcaaaagaccaacaatgctatttcaagtaagcgagaaatacttcgtagattaaatgaaaatcttaaagctcaagaagatgaaaaaggaaagcagaatctctctgatacttttgagataaatgttcatgaagatgccaaagagcatgaaaaagaaaaatcagtttcatctgatcgcaagaagtgggaggcaggaggtcaacttgtgattcctctggatgagttaacactagatacatccttctctacaactgaaagacatacagtgggagaagttattaaattaggtcctaatggatctccaagaagagcctgggggaaaagtccgacagattctgttctaaagatacttggagaagctgaactacaacttcagacagaactattagaaaatacaactattagaagtgagatttctcccgaaggggaaaagtacaaacccttaattactggagaaaaaaaagtacaatgtatttcacatgaaataaacccatcagctattgttgattctcctgttgagacaaaaagtcccgagttcagtgaggcatctccacagatgtcattgaaactggaaggaaatttagaagaacctgatgatttggaaacagaaattctacaagagccaagtggaacaaacaaagatgagagcttgccatgcactattactgatgtgtggattagtgaggaaaaagaaacaaaggaaactcagtcggcagataggatcaccattcaggaaaatgaagtttctgaagatggagtctcgagtactgtggaccaacttagtgacattcatatagagcctggaaccaatgattctcagcactctaaatgtgatgtagataagtctgtgcaaccggaaccatttttccataaggtggttcattctgaacacttgaacttagtccctcaagttcaatcagttcagtgttcaccagaagaatcctttgcatttcgatctcactcgcatttaccaccaaaaaataaaaacaagaattccttgctgattggactttcaactggtctgtttgatgcaaacaacccaaagatgttaaggacatgttcacttccagatctctcaaagctgttcagaacccttatggatgttcccaccgtaggagatgttcgtcaagacaatcttgaaatagatgaaattgaagatgaaaacattaaagaaggaccttctgattctgaagacattgtgtttgaagaaactgacacagatttacaagagctgcaggcctcgatggaacagttacttagggaacaacctggtgaagaatacagtgaagaagaagagtcagtcttgaagaacagtgatgtggagccaactgcaaatgggacagatgtggcagatgaagatgacaatcccagcagtgaaagtgccctgaacgaagaatggcactcagataacagtgatggtgaaattgctagtgaatgtgaatgcgatagtgtctttaaccatttagaggaactgagacttcatctggagcaggaaatgggctttgaaaaattctttgaggtttatgagaaaataaaggctattcatgaagatgaagatgaaaatattgaaatttgttcaaaaatagttcaaaatattttgggaaatgaacatcagcatctttatgccaagattcttcatttagtcatggcagatggagcctaccaagaagataatgatgaataatcctcaaaatgttttttaatcctcaactatatgaaagcatttgaatttggcttatcagaataacaagcttcagtgggaaatacagcaattatttatttaaaaaatcagatttaagatggactttcttattgcatgaaaaagatggagaaacatgccatttttcagtgaagattctaatattttatctattttgttcattgaattccatggttaaatctcataaaatatatactttattaaatcatccaaccaaagcataggaaacattgacccagaacctgacttaatggttttgaagatttactatgcaatagggtaactttgagtttcagcaaatgtctttaggttgaaggaattacctatgtcatgaaggacctgtctgtggtttttcaatggagtctttaagcatgatcttttttctgtctagtacttgttttcattctggccagcagttctacattaaatcaccttgtcaagggctctgtttacatctatacattttgaagatgaaatttttagccttaaagtttatattctcaagtccttttacaatcagtgtgtctcctgaactagcacacaggctgtagaaacagtcttagaaatcattgaaagatttgattatgaaagaatagcaaaattatatttcttgacatataaaaagttggtttaatgcctttatttctctttaaggaccagaaccaggaatactatatcgaaaaattagtctgtggatttaacactgacttagcatatagcttaaagttgctcttttggtttttaacttcctccatacataagcttcaaggacaataagatgttaaaaaggaggaaataattatttttattttgacactgtgacagttttggtaactaggatcctagggagggaaatgtttgcctgttgaacttctttctgttatgagaggatttagttaggtcattaagatgttgatcacacagcttcaatcacaatatgccaagtataacctggtttcgttagaggtgtctacagtccagatgttcttcgtaataaaagcaaagtttttgaacctctgagtccaaagcaggctggttggcataatatgtaatttgaaaaataaaatcttatcttgcagcactatcagtatgttgaatttattatgtatattatttctaatatccgaaactaaatacttgattttttaatatgtgtgtttattttatgatattgctattaaatttttattatctacctgaagtaaaaaaaaaaaaaaaaa

SEQ ID NO: 111 - DNA, 823 bp, Homo sapiens
  Homo sapiens ubiquitin-conjugating enzyme E2C (UBE2C), transcript variant 1, mRNA
aaacgcgggcgggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacagtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttagacaaaaaaaaaaaaaaa

SEQ ID NO: 112 - DNA, 4665 bp, Homo sapiens
  Homo sapiens muscleblind-like 2 (Drosophila) (MBNL2), transcript variant 1, mRNA
tgaaggtaaaattttccagatacggcagacggctttcagagtacaataaacagggaatgagaactatttacatggaagtttctttctcatgatgcggtggagaagcctcggccacttggttctgccagatgttcctggggttactgtaaatgggaaggacaggcagagctaaacaaggtttatcatttaaaagtgcctgtgtgaagtcacttttgctggaaaactgcagcttgggagctttctttgtattcacatcccactcttctgtcaagtacactttaccctgaccttatgagtggatgaagatacctcagttgtctgactttgccaattgcttaatttcagaatttaaaaaggggaaagaaaaacatcctgctaaaatatgaacatctgagtgtcttattttccaacatcgtcaatagctgtgagcgtcagcattaaatattctcccaaggagtgccatgatattgaagtcactttattaataacagctgtatctgcaaaacagtcaagagactcggacgttgaaagccagagatgacactgagcatgcttttattgcggcctaccatctttaagtgggacatattgattgatgagtgattgcctgtccatacactctctcatcatcctgttccttggattggacttcactaagcaatttatcactcaccttcagacttacatgtgggagttttcacaacagtagttttggaatcattagaacttggattgatttcatcatttaacagaaacaaacagcccaaattactttatcaccatggctttgaacgttgccccagtcagagatacaaaatggctgacattagaagtctgcagacagtttcaaagaggaacatgctcacgctctgatgaagaatgcaaatttgctcatccccccaaaagttgtcaggttgaaaatggaagagtaattgcctgctttgattccctaaagggccgttgttcgagagagaactgcaagtatcttcaccctccgacacacttaaaaactcaactagaaattaatggaaggaacaatttgattcagcaaaaaactgcagcagcaatgcttgcccagcagatgcaatttatgtttccaggaacaccacttcatccagtgcccactttccctgtaggtcccgcgatagggacaaatacggctattagctttgctccttacctagcacctgtaacccctggagttgggttggtcccaacggaaattctgcccaccacgcctgttattgttcccggaagtccaccggtcactgtcccgggctcaactgcaactcagaaacttctcaggactgacaaactggaggtatgcagggagttccagcgaggaaactgtgcccggggagagaccgactgccgctttgcacaccccgcagacagcaccatgatcgacacaagtgacaacaccgtaaccgtttgtatggattacataaaggggcgttgcatgagggagaaatgcaaatattttcaccctcctgcacacttgcaggccaaaatcaaagctgcgcagcaccaagccaaccaagctgcggtggccgcccaggcagccgcggccgcggccacagtcatggcctttccccctggtgctcttcatcctttaccaaagagacaagcacttgaaaaaagcaatggtaccagcgcggtctttaaccccagcgtcttgcactaccagcaggctctcaccagcgcacagttgcagcaacacgccgcgttcattccaacagggtcagttttgtgcatgacacccgctaccagtattgtacccatgatgcacagcgctacgtccgccactgtctctgcagcaacaactcctgcaacaagtgtccccttcgcagcaacagccacagccaatcagataattctgaaataatcagcagaaacggaatggaatgccaagaatctgcattgagaataactaaacattgttactgtacatactatcctgtttcctcctcaatagaattgccacaaactgcatgctaaataaagatgtagttcttctggacagaccacaactctaagaagctagtgctgctatctcatatatgagtattaaatatggtatgcttagtatattccaacctaagatagttaactacctgagaccagctgtgatgtttaaagacataaaggataaagtttacttttaaagggtttctaaacatagtttctgtcctaggaatattgtcttatctccataactatagctgatgcagaaagtccagccagtttactcatttcgattcagaatatttcaaatttagcaataaacaattagcattagttaaaaaagaaacatattccaagggcaggttcgattctagctctaattactgtcatgtcatttacccactggatcaaagggtatgtttcacttcttgacaatataaatgctgcagcaaagatgagaggtgaagtaaaaccgatacctgtcctgcaggtctaaaatttgaatggaaattcaagcacaagtactggggacacatcaaagtgtggtgtttggtttgcctggagatgccacgttgaatcatgtgattctagattaacattaaatagattgaaaaagaaactttgcacggtatgagcttcataccccaccaaacaaagtcttgaaggtattattttacaagtatatttttaaagttgttttataagagagactttgtagaagtgcctagattttgccagacttcatccagcttgacaagattgagaggcccatgccaacagtctaatctaagagattagtctttcaaactcaccatccagttgcctgttacagaataactcttcttaactaaaaacctagtcaaacaaggaagctgtaggtgaggagatctgtataatattctaatttaagtaagtttgagtttagtcactgcaaatttgactgtgactttaatctaaattactatgtaaacaaaaagtagatagtttcactttttaaaaaatccattactgttttgcatttcaaaagttggattaaagggttgtaactgactacagcatggaaaaaaatagttcttttaattctttcaccttaaagcatattttatgtctcaaaagtataaaaaactttaatacaagtacatacatattatatatacacatacatatatatactatatatggatgaaacatattttaatgttgtttacttttttaaatacttggttgatcttcaaggtaatagcgatacaattaaattttgttcagaaagtttgttttaaagtttattttaagcactatcgtaccaaatatttcatatttcacattttatatgttgcacatagcctatacagtacctacatagtttttaaattattgtttaaaaaacaaaacagctgttataaatgaatattatgtgtaattgtttcaaacatccattttctttgtgaacatattagtgattgaagtattttgacttttgagattgaatgtaaaatattttaaatttgggatcatcgcctgttctgaaaactagatgcaccaaccgtatcattatttgtttgaggaaaaaaagaaatctgcattttaattcatgttggtcaaagtcgaattactatctatttatcttatatcgtagatctgataaccctatctaaaagaaagtcacacgctaaatgtattcttacatagtgcttgtatcgttgcatttgttttaatttgtggaaaagtattgtatctaacttgtattactttggtagtttcatctttatgtattattgatatttgtaattttctcaactataacaatgtagttacgctacaacttgcctaaaacattcaaacttgttttcttttttctgtttttttctttgttaattcatttaaactcattgaaaacatagtatacattactaaaaggtaaattatgggaatcactgaaatatttttgtagattaattgttgtaacattgtctttcttttttttcttttgtttcatgattttgatttttaaaattattagcacacaactattttcagccctttaataatggagcatcaaaaacatcacctgtaaccccaagcaaatatagaagactgtattttttactatgatatccattttccagaattgtgattacaatatgcaaagagtcataaatatgccatttacaataaggaggaggcaaggcaaatgcatagatgtacaaatatatgtacaacagattttgctttttatttatttataatgtaattttatagaataattctgggatttgagaggatctaaaactatttttctgtataaatattatttgccaaaagtttgtttatattcagaagtctgactatgatgaataaatcttaaatgctttgtttaattaaaaaacaaaaatcaccaatatccaagacatgaagatatcagttcaacaaatactgtagttaagagactaactctccacttgtatgggaactacatttcactcttggttttcaggatataacagcacttcaccgaaatattctttcagccataccactggtaacatttctactaaatctttctgtaacacttaaagaattccctcattcattaccttacagtgtaaacaggagtctaatttgtatcaatactatgttttggttgtaatattcagttcactcacccaatgtacaaccaatgaaataaaagaagcatttaaa

SEQ ID NO: 113 - DNA, 619 bp, Homo sapiens
  Homo sapiens fatty acid binding protein 4, adipocyte (FABP4), mRNA
tgcagcttccttctcaccttgaagaataatcctagaaaactcacaaaatgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccctgatttagcaagcaagtaattttctcccaagctgattttattcaatatggttacgttggttaaataactttttttagatttag

SEQ ID NO: 114 - DNA, 1619 bp, Homo sapiens
  Homo sapiens baculoviral IAP repeat-containing 5 (survivin) (BIRC5), mRNA
ccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttc

SEQ ID NO: 115 - DNA, 5910 bp, Homo sapiens
  Homo sapiens collagen, type XVIII, alpha 1 (COL18A1), transcript variant 1, mRNA
agctccagccgcactgccccgatggctccctacccctgtggctgccacatcctgctgctgctcttctgctgcctggcggctgcccgggccaacctgctgaacctgaactggctttggttcaataatgaggacaccagccacgcagctaccacgatccctgagccccaggggcccctgcctgtgcagcccacagcagataccaccacacacgtgaccccccggaatggttccacagagccagcgacagcccctggcagccctgagccaccctcagagctgctggaagatggccaggacacccccacttctgccgagagcccggacgcgccagaggagaacattgccggtgtcggagccgagatcctgaacgtggccaaaggcatccggagcttcgtccagctgtggaatgacactgtccccactgagagcttggccagggcggaaaccctggtcctggagactcctgtgggcccccttgccctcgctgggccttccagcaccccccaggagaatgggaccactctctggcccagccgtggcattcctagctctccgggcgcccacacaaccgaggctggcaccttgcctgcacccaccccatcgcctccgtccctgggcaggccctgggcaccactcacggggccctcagtgccaccaccatcttcagagcgcatcagcgaggaggtggggctgctgcagctccttggggaccccccgccccagcaggtcacccagacggatgaccccgacgtcgggctggcctacgtctttgggccagatgccaacagtggccaagtggcccggtaccacttccccagcctcttcttccgtgacttctcactgctgttccacatccggccagccacagagggcccaggggtgctgttcgccatcacggactcggcgcaggccatggtcttgctgggcgtgaagctctctggggtgcaggacgggcaccaggacatctccctgctctacacagaacctggtgcaggccagacccacacagccgccagcttccggctccccgccttcgtcggccagtggacacacttagccctcagtgtggcaggtggctttgtggccctctacgtggactgtgaggagttccagagaatgccgcttgctcggtcctcacggggcctggagctggagcctggcgccgggctcttcgtggctcaggcggggggagcggaccctgacaagttccagggggtgatcgctgagctgaaggtgcgcagggacccccaggtgagccccatgcactgcctggacgaggaaggcgatgactcagatggggcattcggagactctggcagcgggctcggggacgcccgggagcttctcagggaggagacgggcgcggccctaaaacccaggctccccgcgccaccccccgtcaccacgccacccttggctggaggcagcagcacggaagattccagaagtgaagaagtcgaggagcagaccacggtggcttcgttaggagctcagacacttcctggctcagattctgtctccacgtgggacgggagtgtccggacccctgggggccgcgtgaaagagggcggcctgaaggggcagaaaggggagccaggtgttccgggcccacctggccgggcaggccccccaggatccccatgcctacctggtcccccgggtctcccgtgcccagtgagtcccctgggtcctgcaggcccagcgttgcaaactgtccccggaccacaaggacccccagggcctccggggagggacggcacccctggaagggacggcgagccgggcgaccccggtgaagacggaaagccgggcgacaccgggccacaaggcttccctgggactccaggggatgtaggtcccaagggagacaagggagaccctggggttggagagagagggcccccaggaccccaagggcctccagggcccccaggaccctccttcagacacgacaagctgaccttcattgacatggagggatctggctttgggggcgatctggaggccctgcggggtcctcgaggcttccctggacctcccggaccccccggtgtcccaggcctgcccggcgagccaggccgctttggggtgaacagctccgacgtcccaggacccgccggccttcctggtgtgcctgggcgcgagggtccccccgggtttcctggcctcccgggacccccaggccctccgggaagagaggggcccccaggaaggactgggcagaaaggcagcctgggtgaagcaggcgccccaggacataaggggagcaagggagcccccggtcctgctggtgctcgtggggagagcggcctggcaggagcccccggacctgctggaccaccaggcccccctgggccccctgggcccccaggaccaggactccccgctggatttgatgacatggaaggctccggggggcccttctggtcaacagcccgaagcgctgatgggccacagggacctcccggcctgccgggacttaagggggatcctggcgtgcctgggctgccgggggcgaagggagaagttggagcagatggaatccccgggttccccggcctccctggcagagagggcattgctgggccccaggggccaaagggagacagaggcagccggggagaaaagggagatccagggaaggacggagtcgggcagccgggcctccctggcccccccggacccccgggacctgtggtctacgtgtcggagcaggacggatccgtcctgagcgtgccgggacctgagggccggccgggtttcgcaggctttcccggacctgcaggacccaagggcaacctgggctctaagggcgaacgaggctccccgggacccaagggtgagaagggtgaaccgggcagcatcttcagccccgacggcggtgccctgggccctgcccagaaaggagccaagggagagccgggcttccgaggacccccgggtccatacggacggccggggtacaagggagagattggctttcctggacggccgggtcgccccgggatgaacggattgaaaggagagaaaggggagccgggagatgccagccttggatttggcatgaggggaatgcccggccccccaggacctccagggcccccaggccctccagggactcctgtttacgacagcaatgtgtttgctgagtccagccgccccgggcctccaggattgccagggaatcagggccctccaggacccaagggcgccaaaggagaagtgggcccccccggaccaccagggcagtttccgtttgactttcttcagttggaggctgaaatgaagggggagaagggagaccgaggtgatgcaggacagaaaggcgaaaggggggagcccgggggcggcggtttcttcggctccagcctgcccggcccccccggccccccaggcccacgtggctaccctgggattccaggtcccaagggagagagcatccggggccagcccggcccacctggacctcagggaccccccggcatcggctacgaggggcgccagggccctcccggccccccaggccccccagggcccccttcatttcctggccctcacaggcagactatcagcgttcccggccctccgggcccccctgggccccctgggccccctggaaccatgggcgcctcctcaggggtgaggctctgggctacacgccaggccatgctgggccaggtgcacgaggttcccgagggctggctcatcttcgtggccgagcaggaggagctctacgtccgcgtgcagaacgggttccggaaggtccagctggaggcccggacaccactcccacgagggacggacaatgaagtggccgccttgcagccccccgtggtgcagctgcacgacagcaacccctacccgcggcgggagcacccccaccccaccgcgcggccctggcgggcagatgacatcctggccagcccccctcgcctgcccgagccccagccctaccccggagccccgcaccacagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagccaccgcctggatgcggatggccggagaggaccggcggctcggaggaagcccccaccgtgggcagggagcggccggccagcccctggccccaggacctggctgccatactttcctgtatagttcacgtttcatgtaatcctcaagaaataaaaggaagccaaagagtgtatttttttaaaagtttaaaacagaagcctgatgctgacattcacctgccccaactctcccctgacctgtgagcccagctgggtcaggcagggtgcagtatcatgccctgtgcaacctcttggcctgatcagaccacggctcgatttctccaggatttcctgctttgggaagccgtgctcgccccagcaggtgctgacttcatctcccacctagcagcaccgttctgtgcacaaaacccagacctgttagcagacaggccccgtgaggcaatgggagctgaggccacactcagcacaaggccatctgggctcctccagggtgtgtgctcgccctgcggtagatgggagggaggctcaggtccctggggctagggggagccccttctgctcagctctgggccattctccacagcaaccccaggctgaagcaggttcccaagctcagaggcgcactgtgacccccagctccggcctgtcctccaacaccaagcacagcagcctggggctggcctcccaaatgagccatgagatgatacatccaaagcagacagctccaccctggccgagtccaagctgggagattcaagggacccatgagttggggtctggcagcctcccatccagggcccccatctcatgcccctggctgggacgtggctcagccagcacttgtccagctgagcgccaggatggaacacggccacatcaaagaggctgaggctggcacaggacatgcggtagccagcacacagggcagtgagggagggctgtcatctgtgcactgcccatggacaggctggctccagatgcagggcagtcattggctgtctcctaggaaacccatatccttaccctccttgggactgaaggggaaccccggggtgcccacaggccgccctgcgggtgaacaaagcagccacgaggtgcaacaaggtcctctgtcagtcacagccacccctgagatccggcaacatcaacccgagtcattcgttctgtggagggacaagtggactcagggcagcgccaggctgaccacagcacagccaacacgcacctgcctcaggactgcgacgaaaccggtggggctggttctgtaattgtgtgtgatgtgaagccaattcagacaggcaaataaaagtgaccttttacactgaaaaaaaaaaaaaaaaa

SEQ ID NO: 116 - DNA, 1798 bp, Homo sapiens
  Homo sapiens actin, alpha 2, smooth muscle, aorta (ACTA2), transcript variant 1, mRNA
ctctccccgcccccgcggggcggcgcgcactcacccacccgcgccggagcggacctttggcttggcttgtcagggcttgtccaggagttccgctcctctctccaaccggggtccccctccagcgaccctaaagcttcccagacttccgcttcaattcctgtccgcaccccacgcccacctcaacgtggagcgcagtggtctccgaggagcgccggagctgccccgcctgcccagcggggtcagcacttcgcatcaaggcccaagaaaagcaagtcctccagcgttctgagcacccgggcctgagggaaggtcctaacagcccccgggagccagtctccaacgcctcccgcagcagcccgccgctcccaggtgcccgcgtgcgccgctgccgccgcaatcccgcacgcgtcccgcgcccgccccactttgcctatccccgggactaagacgggaatcctgtgaagcagctccagctatgtgtgaagaagaggacagcactgccttggtgtgtgacaatggctctgggctctgtaaggccggctttgctggggacgatgctcccagggctgttttcccatccattgtgggacgtcccagacatcagggggtgatggtgggaatgggacaaaaagacagctacgtgggtgacgaagcacagagcaaaagaggaatcctgaccctgaagtacccgatagaacatggcatcatcaccaactgggacgacatggaaaagatctggcaccactctttctacaatgagcttcgtgttgcccctgaagagcatcccaccctgctcacggaggcacccctgaaccccaaggccaaccgggagaaaatgactcaaattatgtttgagactttcaatgtcccagccatgtatgtggctatccaggcggtgctgtctctctatgcctctggacgcacaactggcatcgtgctggactctggagatggtgtcacccacaatgtccccatctatgagggctatgccttgccccatgccatcatgcgtctggatctggctggccgagatctcactgactacctcatgaagatcctgactgagcgtggctattccttcgttactactgctgagcgtgagattgtccgggacatcaaggagaaactgtgttatgtagctctggactttgaaaatgagatggccactgccgcatcctcatcctcccttgagaagagttacgagttgcctgatgggcaagtgatcaccatcggaaatgaacgtttccgctgcccagagaccctgttccagccatccttcatcgggatggagtctgctggcatccatgaaaccacctacaacagcatcatgaagtgtgatattgacatcaggaaggacctctatgctaacaatgtcctatcagggggcaccactatgtaccctggcattgccgaccgaatgcagaaggagatcacggccctagcacccagcaccatgaagatcaagatcattgcccctccggagcgcaaatactctgtctggatcggtggctccatcctggcctctctgtccaccttccagcagatgtggatcagcaaacaggaatacgatgaagccgggccttccattgtccaccgcaaatgcttctaaaacactttcctgctcctctctgtctctagcacacaactgtgaatgtcctgtggaattatgccttcagttcttttccaaatcattcctagccaaagctctgactcgttacctatgtgttttttaataaatctgaaataggctactggtaaaaaaaaaaaaaaaaaaaaaaaaaaaaaaaaaaaaaaaaaa

SEQ ID NO: 117 - DNA, 3879 bp, Homo sapiens
  Homo sapiens moesin (MSN), mRNA
ggcacgaggccagccgaatccaagccgtgtgtactgcgtgctcagcactgcccgacagtcctagctaaacttcgccaactccgctgcctttgccgccaccatgcccaaaacgatcagtgtgcgtgtgaccaccatggatgcagagctggagtttgccatccagcccaacaccaccgggaagcagctatttgaccaggtggtgaaaactattggcttgagggaagtttggttctttggtctgcagtaccaggacactaaaggtttctccacctggctgaaactcaataagaaggtgactgcccaggatgtgcggaaggaaagccccctgctctttaagttccgtgccaagttctaccctgaggatgtgtccgaggaattgattcaggacatcactcagcgcctgttctttctgcaagtgaaagagggcattctcaatgatgatatttactgcccgcctgagaccgctgtgctgctggcctcgtatgctgtccagtctaagtatggcgacttcaataaggaagtgcataagtctggctacctggccggagacaagttgctcccgcagagagtcctggaacagcacaaactcaacaaggaccagtgggaggagcggatccaggtgtggcatgaggaacaccgtggcatgctcagggaggatgctgtcctggaatatctgaagattgctcaagatctggagatgtatggtgtgaactacttcagcatcaagaacaagaaaggctcagagctgtggctgggggtggatgccctgggtctcaacatctatgagcagaatgacagactaactcccaagataggcttcccctggagtgaaatcaggaacatctctttcaatgataagaaatttgtcatcaagcccattgacaaaaaagccccggacttcgtcttctatgctccccggctgcggattaacaagcggatcttggccttgtgcatggggaaccatgaactatacatgcgccgtcgcaagcctgataccattgaggtgcagcagatgaaggcacaggcccgggaggagaagcaccagaagcagatggagcgtgctatgctggaaaatgagaagaagaagcgtgaaatggcagagaaggagaaagagaagattgaacgggagaaggaggagctgatggagaggctgaagcagatcgaggaacagactaagaaggctcagcaagaactggaagaacagacccgtagggctctggaacttgagcaggaacggaagcgtgcccagagcgaggctgaaaagctggccaaggagcgtcaagaagctgaagaggccaaggaggccttgctgcaggcctcccgggaccagaaaaagactcaggaacagctggccttggaaatggcagagctgacagctcgaatctcccagctggagatggcccgacagaagaaggagagtgaggctgtggagtggcagcagaaggcccagatggtacaggaagacttggagaagacccgtgctgagctgaagactgccatgagtacacctcatgtggcagagcctgctgagaatgagcaggatgagcaggatgagaatggggcagaggctagtgctgacctacgggctgatgctatggccaaggaccgcagtgaggaggaacgtaccactgaggcagagaagaatgagcgtgtgcagaagcacctgaaggccctcacttcggagctggccaatgccagagatgagtccaagaagactgccaatgacatgatccatgctgagaacatgcgactgggccgagacaaatacaagaccctgcgccagatccggcagggcaacaccaagcagcgcattgacgaatttgagtctatgtaatgggcacccagcctctagggacccctcctccctttttccttgtccccacactcctacacctaactcacctaactcatactgtgctggagccactaactagagcagccctggagtcatgccaagcatttaatgtagccatgggaccaaacctagccccttagcccccacccacttccctgggcaaatgaatggctcactatggtgccaatggaacctcctttctcttctctgttccattgaatctgtatggctagaatatcctacttctccagcctagaggtactttccacttgattttgcaaatgcccttacacttactgttgtcctatgggagtcaagtgtggagtaggttggaagctagctcccctcctctcccctccactgtcttcttcaggtcctgagattacacggtggagtgtatgcggtctaggaatgagacaggacctagatatcttctccagggatgtcaactgacctaaaatttgccctcccatcccgtttagagttatttaggctttgtaacgattgggggaataaaaagatgttcagtcatttttgtttctacctcccagatcggatctgttgcaaactcagcctcaataagccttgtcgttgactttagggactcaatttctccccagggtggatgggggaaatggtgccttcaagaccttcaccaaacatactagaagggcattggccattctattgtggcaaggctgagtagaagatcctaccccaattccttgtaggagtataggccggtctaaagtgagctctatgggcagatctaccccttacttattattccagatctgcagtcacttcgtgggatctgcccctccctgcttcaatacccaaatcctctccagctataacagtagggatgagtacccaaaagctcagccagccccatcaggactcttgtgaaaagagaggatatgttcacacctagcgtcagtattttccctgctaggggttttaggtctcttcccctctcagagctacttgggccatagctcctgctccacagccatcccagccttggcatctagagcttgatgccagtaggctcaactagggagtgagtgcaaaaagctgagtatggtgagagaagcctgtgccctgatccaagtttactcaaccctctcaggtgaccaaaatccccttctcatcactcccctcaaagaggtgactgggccctgcctctgtttgacaaacctctaacccaggtcttgacaccagctgttctgtcccttggagctgtaaaccagagagctgctgggggattctggcctagtcccttccacacccccaccccttgctctcaacccaggagcatccacctccttctctgtctcatgtgtgctcttcttctttctacagtattatgtactctactgatatctaaatattgatttctgccttccttgctaatgcaccattagaagatattagtcttggggcaggatgattttggcctcattactttaccacccccacacctggaaagcatatactatattacaaaatgacattttgccaaaattattaatataagaagctttcagtattagtgatgtcatctgtcactataggtcatacaatccattcttaaagtacttgttatttgtttttattattactgtttgtcttctccccagggttcagtccctcaaggggccatcctgtcccaccatgcagtgccccctagcttagagcctccctcaattccccctggccaccaccccccactctgtgcctgaccttgaggagtcttgtgtgcattgctgtgaattagctcacttggtgatatgtcctatattggctaaattgaaacctggaattgtggggcaatctattaatagctgccttaaagtcagtaacttacccttagggaggctgggggaaaaggttagattttgtattcaggggttttttgtgtactttttgggtttttaaaaaattgtttttggaggggtttatgctcaatccatgttctatttcagtgccaataaaatttaggtgacttcaaaaaaaaaaaa

SEQ ID NO: 118 - DNA, 1976 bp, Homo sapiens
  Homo sapiens karyopherin alpha 2 (RAG cohort 1, importin alpha 1) (KPNA2), mRNA
gccacacggtctttgagctgagtcgaggtggaccctttgaacgcagtcgccctacagccgctgattccccccgcatcgcctcccgtggaagcccaggcccgcttcgcagctttctccctttgtctcataaccatgtccaccaacgagaatgctaatacaccagctgcccgtcttcacagattcaagaacaagggaaaagacagtacagaaatgaggcgtcgcagaatagaggtcaatgtggagctgaggaaagctaagaaggatgaccagatgctgaagaggagaaatgtaagctcatttcctgatgatgctacttctccgctgcaggaaaaccgcaacaaccagggcactgtaaattggtctgttgatgacattgtcaaaggcataaatagcagcaatgtggaaaatcagctccaagctactcaagctgccaggaaactactttccagagaaaaacagccccccatagacaacataatccgggctggtttgattccgaaatttgtgtccttcttgggcagaactgattgtagtcccattcagtttgaatctgcttgggcactcactaacattgcttctgggacatcagaacaaaccaaggctgtggtagatggaggtgccatcccagcattcatttctctgttggcatctccccatgctcacatcagtgaacaagctgtctgggctctaggaaacattgcaggtgatggctcagtgttccgagacttggttattaagtacggtgcagttgacccactgttggctctccttgcagttcctgatatgtcatctttagcatgtggctacttacgtaatcttacctggacactttctaatctttgccgcaacaagaatcctgcacccccgatagatgctgttgagcagattcttcctaccttagttcggctcctgcatcatgatgatccagaagtgttagcagatacctgctgggctatttcctaccttactgatggtccaaatgaacgaattggcatggtggtgaaaacaggagttgtgccccaacttgtgaagcttctaggagcttctgaattgccaattgtgactcctgccctaagagccatagggaatattgtcactggtacagatgaacagactcaggttgtgattgatgcaggagcactcgccgtctttcccagcctgctcaccaaccccaaaactaacattcagaaggaagctacgtggacaatgtcaaacatcacagccggccgccaggaccagatacagcaagttgtgaatcatggattagtcccattccttgtcagtgttctctctaaggcagattttaagacacaaaaggaagctgtgtgggccgtgaccaactataccagtggtggaacagttgaacagattgtgtaccttgttcactgtggcataatagaaccgttgatgaacctcttaactgcaaaagataccaagattattctggttatcctggatgccatttcaaatatctttcaggctgctgagaaactaggtgaaactgagaaacttagtataatgattgaagaatgtggaggcttagacaaaattgaagctctacaaaaccatgaaaatgagtctgtgtataaggcttcgttaagcttaattgagaagtatttctctgtagaggaagaggaagatcaaaacgttgtaccagaaactacctctgaaggctacactttccaagttcaggatggggctcctgggacctttaacttttagatcatgtagctgagacataaatttgttgtgtactacgtttggtattttgtcttattgtttctctactaagaactctttcttaaatgtggtttgttactgtagcactttttacactgaaactatacttgaacagttccaactgtacatacatactgtatgaagcttgtcctctgactaggtttctaatttctatgtggaatttcctatcttgcagcatcctgtaaataaacattcaagtccacccttaaaaaaaa

SEQ ID NO: 119 - DNA, 5494 bp, Homo sapiens
  Homo sapiens collagen, type IV, alpha 3 (Goodpasture antigen) binding protein (COL4A3BP), transcript variant 3, mRNA
aaacgaagcccacccaccgactgacaaggccccaaggggacaagcgatccccgcgcgggatactcacccgttacctcaggatcgcgactacaactcccaggaggctgcgcgagcgacggaccaacgcccttcccagaatgcagcacagctgcatccctaccccgccctctcctttctccgctcctcctgcttttctacccgtcgtcacccgggagagccggagggggctaagttcgggtggcagcgccgggcgcaacgcaggggtcacggcgacggcggcggcggctgacggctggaagggtaggcttccttcaccgctcgtcctccttcctcgctccgctcggtgtcaggcgcggcggcggcgcggcgggcggacttcgtccctcctcctgctcccccccacaccggagcgggcactcttcgcttcgccatcccccgacccttcaccccgaggactgggcgcctcctccggcgcagctgagggagcgggggccggtctcctgctcggttgtcgagcctccatgtcggataatcagagctggaactcgtcgggctcggaggaggatccagagacggagtctgggccgcctgtggagcgctgcggggtcctcagtaagtggacaaactacattcatgggtggcaggatcgttgggtagttttgaaaaataatgctctgagttactacaaatctgaagatgaaacagagtatggctgcagaggatccatctgtcttagcaaggctgtcatcacacctcacgattttgatgaatgtcgatttgatattagtgtaaatgatagtgtttggtatcttcgtgctcaggatccagatcatagacagcaatggatagatgccattgaacagcacaagactgaatctggatatggatctgaatccagcttgcgtcgacatggctcaatggtgtccctggtgtctggagcaagtggctactctgcaacatccacctcttcattcaagaaaggccacagtttacgtgagaagttggctgaaatggaaacatttagagacatcttatgtagacaagttgacacgctacagaagtactttgatgcctgtgctgatgctgtctctaaggatgaacttcaaagggataaagtggtagaagatgatgaagatgactttcctacaacgcgttctgatggtgacttcttgcatagtaccaacggcaataaagaaaagttatttccacatgtgacaccaaaaggaattaatggtatagactttaaaggggaagcgataacttttaaagcaactactgctggaatccttgcaacactttctcattgtattgaactaatggttaaacgtgaggacagctggcagaagagactggataaggaaactgagaagaaaagaagaacagaggaagcatataaaaatgcaatgacagaacttaagaaaaaatcccactttggaggaccagattatgaagaaggccctaacagtctgattaatgaagaagagttctttgatgctgttgaagctgctcttgacagacaagataaaatagaagaacagtcacagagtgaaaaggtgagattacattggcctacatccttgccctctggagatgccttttcttctgtggggacacatagatttgtccaaaagccctatagtcgctcttcctccatgtcttccattgatctagtcagtgcctctgatgatgttcacagattcagctcccaggttgaagagatggtgcagaaccacatgacttactcattacaggatgtaggcggagatgccaattggcagttggttgtagaagaaggagaaatgaaggtatacagaagagaagtagaagaaaatgggattgttctggatcctttaaaagctacccatgcagttaaaggcgtcacaggacatgaagtctgcaattatttctggaatgttgacgttcgcaatgactgggaaacaactatagaaaactttcatgtggtggaaacattagctgataatgcaatcatcatttatcaaacacacaagagggtgtggcctgcttctcagcgagacgtattatatctttctgtcattcgaaagataccagccttgactgaaaatgaccctgaaacttggatagtttgtaatttttctgtggatcatgacagtgctcctctaaacaaccgatgtgtccgtgccaaaataaatgttgctatgatttgtcaaaccttggtaagcccaccagagggaaaccaggaaattagcagggacaacattctatgcaagattacatatgtagctaatgtgaaccctggaggatgggcaccagcctcagtgttaagggcagtggcaaagcgagagtatcctaaatttctaaaacgttttacttcttacgtccaagaaaaaactgcaggaaagcctattttgttctagtattaacagtgactgaagcaaggctgtgtgacattccatgttggagaaaaaaagaaaaaaaaaagctgaatgctctaagctggaacgtaggatctatagccttgtctgtggcccaagaccttggccttgtgtacaaaaatgacaaaatattgcaatagcaaagctgaacatctaacactagctatctcttgctagatctccttgctcagcatataactataaatacatgtaaaattacatgtatatggctatatttttatttgcttgctcctagaagagaaaaaaaaatcaactttgaatcacaactaggaattgatgctttaatttttggatactttttcagaatttttaatttactatggtccggcctaagatcctctgttgtatcaggttttgtgcacaaaagaaaagcacaaaagttgaatgcacatggggcatgtgctttctgtgcaccaaatatctggatgaggttcttttttcaggcctacagtcaaatctgtgtccagaattttttgacttttttgctttgtataatcatagaattcattgctgctgatttctataatgattcatgttgtcatgtgtctcttaataactgagggctgtcagtaacctgtgattttgccttttctatagtcttactcccatgaagaaccttggttctgatggagaaagtgaaaagctttatttcttcccctagatatctttatatttctattatattttttagttgtgtactgtgtactagagatttttttcagtttgttatgaacacaatttggtaagccctaaattggttctgcctgtctccaaacagaaacatctgtacaaatcttgttggtatagactactttctggaaaatggtcaagataagttcatgttttcttgaaatttctaagatagtatatggtatcacttgtttaaagcaaatcagactgagtttgacatttaattcaatatttctggtattcagtaacgggtatatatgtttgttcttccagtttgggtcagtttaaaagatatgttgcaaagtatacatagaaaatgtgagcaatgcctctctttgccttttgatcagaaacttcagcagagcggtaaggattccacatgatttaaactgaaatgcttttctttgttgctgtaagaacttaaaatgtaaaatacctttttcagtttaagtcctgtaaacaacattgaagcatggagatgaggcaaggaatagtactcactgaagttgaaatgactgcccacttcaaaatcttcattgtgtttacacaccagtgtatttatacaaatcagaggcattttgtagatgctttgctgacttgttcagctctgtaaaaacacagaaatcagacccattttgtaaagcggaaaatcatgttacatggaacatgtcctgtatatatcacatacatggtaatggagtcttaatgataagtgcaagataataatttaatgatgggattagtctgatcgcttaatatgcacaatcctggaagtgaattacttgcatcagatatagtgatatttattattctgtacagagagaaaaatacatataaaacatatgcttacattacatgcacgcggatttcatgctccataatcttttctattttttaatttacctttctgtaaatgatgtgcatggaatatgccttatagaaaaatgctgttcataatttgactacgtggaaaagtgcctatatggtggtaatgctagtaaggcaaataagacaaattatcatgttggtttactacatcaccagttaacattttatattgtgatgtttaaaaaagaaaaatttatacctcaaatgtgtattttattttacaatcagctgtggggtatggggttgggatgggagaatgggggggttggggagggcaggtttattcaccatagccgctgataagaatcttcaaaaaaattctatatgcgcactataaatgtttctctgtttgccatttctggtaactatcatgaacacagacagttaactctttcataactgaattggatagctttattttacagaagtatggcaagtttacaaagcaatatctaaatctaattatcattagttgcatttggactaaatgtgatgatatacttttgcaattgattctgtaaataaaaggattacactaaaatatttgtattaaaagaagaaaagataacattttacctttagataactgcacttgtacctcactagagttaatcccacccaatcagattgagaaataaattggggaatgtggaaagagtccaaaagaggtcagaatttggagaggtactggccttctggacaacatttagaccctctacaattattttcattaagctgattcctacatcctgaatattcatgttttctcatctacagatatttgtcttcccccaaactaaaagaaaaaaaactaccctttactctcttttctactcagttactcttttgtgctatgttagaaacttgaaatatattggtgatgtggggattttgtccctgactgcccactgtacaggacaagagagtacagtgtttcagttggaattcaggactcctggttttgaggtagaggatgatcactgcagtacttggtttggaattgccacaggggtagctaaaccaaaggagggttatatctgcaagggaggtgtaagaaggcaaaataaggaaaaggaggaatgggttttctatttgttcagtttcatcaactaatttatacacttaatacaacttcagtgtcaattgctattaagaaatttttagttgggctgagctggttctcttgtgaaattgtgctggttatctttaagcttatcagttatttgtccaattaaacacttttcaccagtatttagtccgagttgtacagacgatgtatttggattttgtcatggttcatctacagactcaaaacataatcattttaaagtaccttgggagtgtgtagagtaacttctataatagctttatgatcctgatgatgttttttaaacacaataaagttggatcttccatgttacaatcacagaattaaaaccagtatttaaagtggaaaagtattaaaatattatggacaaata

SEQ ID NO: 120 - DNA, 3701 bp, Homo sapiens
  Homo sapiens cell division cycle 25 homolog B (S. pombe) (CDC25B), transcript variant 1, mRNA
gcagccagtcgcggaggcggggaggctgcgcggtcagaggcgcctggagcgagcgaatcctggcccaccgcctgcccaaccgcgtgaccttgattgagttaatgaacttcacgcctcagcgtccaggtctgtaaaatggggtgtctaacgcagaccgtacagcccagctgggtttagcaaacttccgggagccagttggagcctctccccatccctagcggtgatcccaggtgacgacatgccgcggggggtcctgcggaggccaccctagggcgttgctgctgcctttgggagtgtggagctccaaaccatgtcgcgagaggcggattttgggaggccgggatcctcgcgccagggggatgtgcgagggtgtgggataaatcttaattcctccggcccacccaaagcctggaaatccagcctccgcgcctcttgccctgcgggccccgccctcagtcccgccctcatctaacccgctaccccattggtggcgtccggcggcgcggctgctgttatttttcgaatatataaggaggtggaagtggcagctgcaactagaggcttccctggctggtgcctgagcccggcgtccctcgccccccgccctccccgcatccctctcctccctcgcgcctggccctgtggctcttcctccctccctccttccccccccccccacccctcgcccgctgcctccctcggcccagccagctgtgccggcgtttgttggctgccctgcgcccggccctccagccagccttctgccggccccgccgcgatggaggtgccccagccggagcccgcgccaggctcggctctcagtccagcaggcgtgtgcggtggcgcccagcgtccgggccacctcccgggcctcctgctgggatctcatggcctcctggggtccccggtgcgggcggccgcttcctcgccggtcaccaccctcacccagaccatgcacgacctcgccgggctcggcagcgaaaccccaaagagtcaggtagggaccctgctcttccgcagccgcagccgcctgacgcacctatccctgtctcgacgggcatccgaatcctccctgtcgtctgaatcctccgaatcttctgatgcaggtctctgcatggattcccccagccctatggacccccacatggcggagcagacgtttgaacaggccatccaggcagccagccggatcattcgaaacgagcagtttgccatcagacgcttccagtctatgccggtgaggctgctgggccacagccccgtgcttcggaacatcaccaactcccaggcgcccgacggccggaggaagagcgaggcgggcagtggagctgccagcagctctggggaagacaaggagaatgatggatttgtcttcaagatgccatggaagcccacacatcccagctccacccatgctctggcagagtgggccagccgcagggaagcctttgcccagagacccagctcggcccccgacctgatgtgtctcagtcctgaccggaagatggaagtggaggagctcagccccctggccctaggtcgcttctctctgacccctgcagagggggatactgaggaagatgatggatttgtggacatcctagagagtgacttaaaggatgatgatgcagttcccccaggcatggagagtctcattagtgccccactggtcaagaccttggaaaaggaagaggaaaaggacctcgtcatgtacagcaagtgccagcggctcttccgctctccgtccatgccctgcagcgtgatccggcccatcctcaagaggctggagcggccccaggacagggacacgcccgtgcagaataagcggaggcggagcgtgacccctcctgaggagcagcaggaggctgaggaacctaaagcccgcgtcctccgctcaaaatcactgtgtcacgatgagatcgagaacctcctggacagtgaccaccgagagctgattggagattactctaaggccttcctcctacagacagtagacggaaagcaccaagacctcaagtacatctcaccagaaacgatggtggccctattgacgggcaagttcagcaacatcgtggataagtttgtgattgtagactgcagatacccctatgaatatgaaggcgggcacatcaagactgcggtgaacttgcccctggaacgcgacgccgagagcttcctactgaagagccccatcgcgccctgtagcctggacaagagagtcatcctcattttccactgtgaattctcatctgagcgtgggccccgcatgtgccgtttcatcagggaacgagaccgtgctgtcaacgactaccccagcctctactaccctgagatgtatatcctgaaaggcggctacaaggagttcttccctcagcacccgaacttctgtgaaccccaggactaccggcccatgaaccacgaggccttcaaggatgagctaaagaccttccgcctcaagactcgcagctgggctggggagcggagccggcgggagctctgtagccggctgcaggaccagtgaggggcctgcgccagtcctgctacctcccttgcctttcgaggcctgaagccagctgccctatgggcctgccgggctgagggcctgctggaggcctcaggtgctgtccatgggaaagatggtgtgggtgtcctgcctgtctgccccagcccagattcccctgtgtcatcccatcattttccatatcctggtgccccccacccctggaagagcccagtctgttgagttagttaagttgggttaataccagcttaaaggcagtattttgtgtcctccaggagcttcttgtttccttgttagggttaacccttcatcttcctgtgtcctgaaacgctcctttgtgtgtgtgtcagctgaggctgggggagagccgtggtccctgaggatgggtcagagctaaactccttcctggcctgagagtcagctctctgccctgtgtacttcccgggccagggctgcccctaatctctgtaggaaccgtggtatgtctgccatgttgcccctttctcttttcccctttcctgtcccaccatacgagcacctccagcctgaacagaagctcttactctttcctatttcagtgttacctgtgtgcttggtctgtttgactttacgcccatctcaggacacttccgtagactgtttaggttcccctgtcaaatatcagttacccactcggtcccagttttgttgccccagaaagggatgttattatccttgggggctcccagggcaagggttaaggcctgaatcatgagcctgctggaagcccagcccctactgctgtgaaccctggggcctgactgctcagaacttgctgctgtcttgttgcggatggatggaaggttggatggatgggtggatggccgtggatggccgtggatgcgcagtgccttgcatacccaaaccaggtgggagcgttttgttgagcatgacagcctgcagcaggaatatatgtgtgcctatttgtgtggacaaaaatatttacacttagggtttggagctattcaagaggaaatgtcacagaagcagctaaaccaaggactgagcaccctctggattctgaatctcaagatgggggcagggctgtgcttgaaggccctgctgagtcatctgttagggccttggttcaataaagcactgagcaagttgagaaaaaaaaaaaaaaaaaaaaaaaaaaaaa